(19) United States
(12) Patent Application Publication
(10) Pub. No.: US 2012/0177629 A1
(43) Pub. Date: Jul. 12, 2012

(54) Predigested Nutritional Formula

(75) Inventors: Delma Broussard, Swarthmore, PA (US); Luigi Ghidorsi, Milano (IT); Giovanni Ortenzi, Monza (IT)

(73) Assignee: Aptalis Pharma Limited, Wicklow (IE)

(21) Appl. No.: 13/205,221

(22) Filed: Aug. 8, 2011

Related U.S. Application Data
(60) Provisional application No. 61/371,608, filed on Aug. 6, 2010.
(60) Provisional application No. 61/470,094, filed on Mar. 31, 2011.

(51) Int. Cl. A61K 38/54; A61P 3/02; C12P 21/06

(52) U.S. Cl. 424/94.21; 435/68.1

(57) ABSTRACT

The present invention is directed to a process for the preparation of a predigested nutritional formula as well as the predigested liquid nutritional formula and a kit for the preparation of a predigested nutritional formula. The predigested nutritional formula comprises digestive enzymes and a liquid nutritional composition comprising a mixture of carbohydrates, lipids, proteins and water.

### CROSS REFERENCE TO RELATED APPLICATIONS

The present application claims priority to U.S. Provisional Application No. 61/371,608 filed Aug. 6, 2010 and U.S. Provisional Application No. 61/470,094 filed Mar. 31, 2011, each of which is incorporated herein by reference in its entirety for all purposes.

### FIELD OF THE INVENTION

The present invention is directed to a predigested nutritional formula. The invention is also directed to a process for the preparation of a predigested nutritional formula comprising mixing digestive enzymes and a liquid nutritional composition comprising a mixture of carbohydrates, lipids, proteins and water to form the predigested nutritional formula.

### BACKGROUND OF THE INVENTION

The proper dosing of medication for patients is an important concern within the medical field. For infants, smaller children, and geriatric patients in particular, as well as sometimes also adult populations, the administration of medications and dosing methods often present substantial issues. As is well known in the art, medications are provided in many forms (e.g., liquid, solid, and combinations of solids in liquids) and are delivered to patients in many ways (e.g., orally, via injection, transdermally).

In cases of exocrine pancreatic insufficiency (EPI), of which the FDA estimates that more than 200,000 Americans suffer, patients are incapable of properly digesting food due to a lack of digestive enzymes made by their pancreas. That loss of digestive enzymes leads to disorders such as the maldigestion and malabsorption of nutrients, which lead to malnutrition and other consequent undesirable physiological conditions associated therewith. These disorders are common for those suffering from cystic fibrosis (CF) and other conditions compromising the exocrine function of the pancreas, such as pancreatic cancer, pancreatectomy, and pancreatitis. This malnutrition can be life threatening if left untreated, particularly in the case of infants and CF patients, and the disorders lead to impaired growth in children, compromised immune response, and shortened life expectancy.

Digestive enzymes, such as pancrelipase and other pancreatic enzymes products (PEPs) can be administered to at least partially remedy EPI. The administration of digestive enzyme supplements allows patients to more effectively digest their food.

Capsules containing digestive enzymes such as pancrelipase (Zenpep®, Creon® and Pancreaze®) have been developed for oral administration. However, if a patient is unable to swallow the capsules, each capsule can be opened and the contents sprinkled on a small amount of food, usually a soft, acidic food (such as commercially available applesauce) and administered orally to the patient with a spoon. Alternatively such medications may be administered orally for infants and children, using a syringe device containing the contents suspended in a medium amenable to administration thereby.

It is also recognized that for some patients, including pediatric and adult patients with EPI, enteral feeding through gastrostomy tubes and smaller lumen enteral feeding tubes, such as nasogastric and jejunal feeding tubes is required. There is therefore a clear need for the administration of digestive enzymes such as pancrelipase to such patients who are unable to take digestive enzymes orally. Furthermore, where the digestive enzymes are in particulate form and are added into a nutritional formula for administration, issues include how to ensure that the digestive enzymes effectively exert their enzyme activity on constituents susceptible thereto in the nutrient formula and to obviate potential obstructions to enteral feeding of by the particulates.

Pancrelipase used for treating EPI is mainly a combination of three enzyme classes: lipase, protease and amylase, together with their various co-factors and co-enzymes. These enzymes are produced naturally in the pancreas and are important in the digestion of fats, proteins and carbohydrates. Pancrelipase is typically prepared from porcine pancreatic glands, although other sources can also be used, for example those described in U.S. Pat. No. 6,051,220, U.S. 2004/0057944, 2001/0046493, and WO2006044529, each of which is herein incorporated by reference in its entirety for all purposes. The enzymes catalyze the hydrolysis of fats into glycerol and fatty acids, starch into dextrin and sugars, and protein into amino acids and derived substances.

Pancreatic enzymes show optimal activity under near neutral and slightly alkaline conditions. Under gastric conditions, pancreatic enzymes may be inactivated with a resulting loss in biological activity. Therefore, exogenously administered enzymes are generally protected against gastric inactivation and remain intact during their transit through the stomach and into the duodenum. Therefore, it is desirable to coat pancreatic enzymes. Pancreatic lipases are the most sensitive to gastric inactivation and are key enzymes in the treatment of malabsorption. Lipase activity is typically monitored to determine the stability of an enzyme composition containing lipase. The entire contents of U.S. Pat. No. 7,658,918 issued to Ortenzi et al. is expressly incorporated by reference in its entirety herein for all purposes, and describes stable digestive enzyme compositions and explains that certain particulate medications, administered orally, are designed to pass through the stomach of the patient and thereafter to release within the intestines. The administration of a proper dosage of such particulate medications to patients, particularly infants and children, should be as accurate as possible.

In view of the aforesaid, there is a need for a practical, inexpensive, simple and effective process for preparing a predigested nutritional formulation; more particularly to one that has been effectively digested and that would be capable of enteral administration without any, or limited susceptibility to obstruction of an enteral feeding tube.

### BRIEF SUMMARY OF THE INVENTION

The invention is directed to a method of preparing a predigested nutritional formulation comprising mixing digestive enzymes and a liquid nutritional composition in order to achieve the predigestion of the liquid nutritional composition prior to its enteral administration to a patient that would benefit from such. The invention is also directed to a predigested nutritional formulation. It is to be understood that both the foregoing general description and the following detailed description are exemplary, but are not restrictive, of the invention.

### BRIEF DESCRIPTION OF THE FIGURES

The invention is best understood from the following detailed description when read in connection with the accompanying following figures:

FIG. 1. Liquid meal+pancrelipase MCT before blending

FIG. 2. Liquid meal+pancrelipase MTC after 1-min blending at 16,500 rpm

FIG. 3. Residue on the glass filter crucible after filtration of liquid meal+pancrelipase MCT homogenate (1-min blending at 16,500 rpm)

FIG. 4. Liquid meal+pancrelipase MTC after 1-min blending at 15,500 rpm

FIG. 5. Residue on the glass filter crucible after filtration of liquid meal (Ensure Plus®)+pancrelipase MTC homogenate (1-min blending at 15,500 rpm)

FIG. 6. Liquid meal+pancrelipase MTC after 2-min blending at 15,500 rpm

FIG. 7. Residue on the glass filter crucible after filtration of liquid meal (Ensure Plus®)+pancrelipase MTC homogenate (2-min blending at 15,500 rpm)

FIG. 8. Graphical representation of the lipase release and stability in liquid meal.

FIG. 9. pH and temperature kinetics during lipolysis

FIG. 10: Plot of the concentration of each lipolysis product in the Exp. 3

FIG. 11. TAG kinetic in Experiment 3

FIG. 12. lipolysis is level 1 (L1) and level (L2) for pancrelipase MTC

FIG. 13. Pancrelipase microtabs (approx. 5,000 lipase UPS units) in 5 mL 8.4% sodium bicarbonate at r.t.—time 0

FIG. 14. Pancrelipase microtabs (approx. 5,000 lipase UPS units) in 5 mL 8.4% sodium bicarbonate at r.t.—10 min

FIG. 15. Pancrelipase microtabs (approx. 5,000 lipase UPS units) in 5 mL 8.4% sodium bicarbonate at r.t.—20 min

FIG. 16. Pancrelipase microtabs (approx. 5,000 lipase UPS units) in 5 mL 8.4% sodium bicarbonate at r.t.—35 min

FIG. 17. Pancrelipase microtabs (approx. 5,000 lipase UPS units) in 5 mL 8.4% sodium bicarbonate at r.t.—45 min

FIG. 18. Pancrelipase microtabs (approx. 5,000 lipase UPS units) in 5 mL 8.4% sodium bicarbonate at r.t.—55 min

FIG. 19. Pancrelipase microtabs (approx. 5,000 lipase UPS units) in 5 mL 8.4% sodium bicarbonate at r.t.—time 0

FIG. 20. Pancrelipase microtabs (approx. 5,000 lipase UPS units) in 5 mL 8.4% sodium bicarbonate at r.t.—10 min

FIG. 21. Pancrelipase microtabs (approx. 5,000 lipase UPS units) in 5 mL 8.4% sodium bicarbonate at r.t.—20 min

FIG. 22. Pancrelipase microtabs (approx. 5,000 lipase UPS units) in 5 mL 8.4% sodium bicarbonate at r.t.—solution stirred after 20-min soaking time

FIG. 23. Pancrelipase minitabs (approx. 5,000 lipase UPS units) in 5 mL 8.4% sodium bicarbonate at r.t.—time 0

FIG. 24. Pancrelipase minitabs (approx. 5,000 lipase UPS units) in 5 mL 8.4% sodium bicarbonate at r.t.—10 min

FIG. 25. Pancrelipase minitabs (approx. 5,000 lipase UPS units) in 5 mL 8.4% sodium bicarbonate at r.t.—20 min

FIG. 26. Pancrelipase minitabs (approx. 5,000 lipase USP units) in 5 mL 8.4% sodium bicarbonate at r.t.—solution stirred after 20 min soaking time

FIG. 27. Pancrelipase minitabs (approx. 5,000 lipase UPS units) in 5 mL 8.4% sodium bicarbonate at r.t.—30 min

FIG. 28. Pancrelipase minitabs (approx. 5,000 lipase UPS units) in 5 mL 8.4% sodium bicarbonate at r.t.—solution stirred at the end of 30-min soaking time

FIG. 29. Pancrelipase microtabs (approx. 5,000 lipase UPS units) in 5 mL 13% sodium bicarbonate at r.t.—time 0

FIG. 30. Pancrelipase microtabs (approx. 5,000 lipase UPS units) in 5 mL 13% sodium bicarbonate at r.t.—10 min

FIG. 31. Pancrelipase microtabs (approx. 5,000 lipase UPS units) in 5 mL 13% sodium bicarbonate at r.t.—20 min

FIG. 32. Pancrelipase microtabs (approx. 5,000 lipase UPS units) in 5 mL 0.65% sodium bicarbonate at r.t.—time 0

FIG. 33. Pancrelipase microtabs (approx. 5,000 lipase UPS units) in 5 mL 0.65% sodium bicarbonate at r.t.—35 min

FIG. 34. Pancrelipase microtabs (approx. 40,000 lipase UPS units U) in 5 mL 8.4% sodium bicarbonate at r.t.—time 0

FIG. 35. Pancrelipase microtabs (approx. 40,000 lipase UPS units) in 5 mL 8.4% sodium bicarbonate at r.t.—20 min

FIG. 36. Pancrelipase microtabs (approx. 40,000 lipase UPS units) in 5 mL 8.4% sodium bicarbonate at r.t.—45 min

FIG. 37. Pancrelipase microtabs (approx. 40,000 lipase UPS units) in 5 mL 8.4% sodium bicarbonate at 4° C.—time 0

FIG. 38. Pancrelipase microtabs (approx. 40,000 lipase UPS units) in 5 mL 8.4% sodium bicarbonate at 4° C.—45 min

FIG. 39. Pancrelipase microtabs (approx. 40,000 lipase UPS units) in 5 mL 8.4% sodium bicarbonate at r.t.—20 min

FIG. 40. Pancrelipase microtabs (approx. 40,000 lipase USP units) in 15 mL 8.4% sodium bicarbonate at r.t.—20 min

FIG. 41. Pancrelipase microtabs (approx. 40,000 lipase USP U) in 25 mL 8.4% sodium bicarbonate at r.t.—20 min

FIG. 42. Pancrelipase pellets (approx 40,000 lipase USP units) in 25 mL 8.4% bicarbonate solution at r.t.—time 0

FIG. 43. Pancrelipase pellets (approx 40,000 lipase USP units) in 25 mL 8.4% bicarbonate solution at r.t.—20 min

FIG. 44. Pancrelipase pellets (approx 40,000 lipase USP units) in 25 mL 8.4% bicarbonate solution at r.t.—90 min

FIG. 45. Pancrelipase pellets (approx 40,000 lipase USP units) in 25 mL 8.4% bicarbonate solution at r.t.—120 min

FIG. 46. Residual lipase activity of 40,000 lipase UPS units, pancrelipase microtabs vs. pancrelipase powder, in 25 mL 8.4% sodium bicarbonate solution stored at r.t./4° C.

### DETAILED DESCRIPTION OF THE INVENTION

The present invention is directed to a process for the preparation of a predigested nutritional formula comprising mixing digestive enzymes or an enzyme solution thereof and a liquid nutritional composition comprising a mixture of carbohydrates, lipids, proteins and water to form the predigested nutritional formula.

In one embodiment of the invention the mixing is preceded by the step of the addition of digestive enzymes or enzyme solution thereof to the liquid nutritional composition.

In another embodiment of the invention the mixing is preceded by the step of the addition of digestive enzymes to the liquid nutritional composition.

In another embodiment of the invention the mixing is preceded by the step of the addition of digestive enzymes solution to the liquid nutritional composition.

In another embodiment of the invention the digestive enzymes are in the form of pancrelipase beads.

In another embodiment of the invention the digestive enzymes are in the form of pancrelipase beads that are enterically coated.

In another embodiment of the invention the digestive enzymes are in the form of pancrelipase beads that are enterically coated and the mixing is conducted by mechanical blending.

In another embodiment of the invention the mixing is conducted by mechanical blending of the pancrelipase beads and liquid nutritional composition until the mixture is homogenized.

In another embodiment of the invention the digestive enzymes are in the form of pancrelipase beads that are enterically coated and that are suspended in a pharmaceutically acceptable weakly basic solution to form the enzyme solution thereof.

The predigested nutritional formula of the present invention can be prepared starting from any suitable oral dosage form that contains digestive enzymes. Non-limiting examples of suitable dosage forms include tablets, capsules, or sachets. In a particular embodiment, the dosage form is capsules. Each dosage form contains digestive enzyme beads of medication. For the present invention the digestive enzyme beads are any kind of particulates that can undergo mechanical mixing or mixing with a pharmaceutically acceptable weakly basic solution in order to release the active enzyme contained herein. The term “bead” includes granules, tablets, spheres, minitablets, microtablets, microparticles, microspheres, microcapsules, micropellets, as well as particles having diameters up to about 5 mm; the bead may be any suitable particle size or shape. For example, the beads can be in the form of a “micropellets” having a particle size range of about 50-5,000 μm, or can be in the form of “minitablets” which have a nominal (e.g., mean) particle diameter in the range of about 2-5 mm. This particulate can be “microtablets” which have nominal (e.g., mean) particle diameters of less than about 2 mm, for example about 1-2 mm. “Minimicrospheres” having the smallest median particles size of 1.15 mm or “microtablets” having highest median particles at 2.63 mm are also suitable for the present process. The particles can be in the form of “microcapsules” having an average particle size of less than about 800 μm, preferably less than 500 μm, preferably of about 400 μm to about 600 μm or of about 250 μm to about 500 μm. These beads may be also “micropellets” having a volume diameter (d(v,0.1) (defined as the diameter where 10% of the volume distribution is below this value and 90% is above this value) of not less than 400 μm and a volume diameter d(v,0.9), (defined as the diameter where 90% of the volume distribution is below this value and 10% is above this value) of not more than 900 μm. The specific surface may range from between 8.7 cm²/g to 19.8 cm²/g.

All the digestive enzyme beads, more particularly pancrelipase enzyme beads, suitable for the preparation of the predigested nutritional formula may be coated by an enteric coating.

The phrase “enteric polymer” means a polymer that protects the digestive enzymes from gastric contents, for example a polymer that is stable at acidic pH, but can break down or dissolve rapidly at higher pH, or a polymer whose rate of hydration or erosion is slow enough to ensure that contact of gastric contents with the digestive enzymes is relatively minor while it is in the stomach, as opposed to the remainder of the gastro-intestinal tract. The enteric polymer is a constituent of the enteric coating which may further include plasticizers and further excipients. Non-limiting examples of enteric polymers include those known in the art, such as modified or unmodified natural polymers such as cellulose acetate phthalate, hydroxypropylmethylcellulose phthalate, hydroxypropylmethylcellulose acetate succinate, and shellac; or synthetic polymers such as acrylic polymers or copolymers methacrylic acid polymers and copolymers, methylmethacrylate copolymers, and methacrylic acid/methylmethacrylate copolymers. The enteric coating encapsulates the core comprised of the delayed release beads of pancrelipase. The coating acts as a barrier protecting the medication from the acidic environment of the stomach and substantially prevents the release of the medication before it reaches the small intestine. The coated stabilized digestive enzyme particles can then be formulated into capsules. A particular dosage form of stabilized digestive enzyme particles is a capsule filled with enteric coated pancrelipase enzymes beads.

The term “digestive enzyme” used herein denotes an enzyme in the alimentary tract which breaks down the components of food so that they can be taken or absorbed by the organism. Non-limiting examples of digestive enzymes include pancrelipase (also referred to as pancreatin), lipase, co-lipase, trypsin, chymotrypsin, chymotrypsin B, pancreatopeptidase, carboxypeptidase A, carboxypeptidase B, glycerol ester hydrolase, phospholipase, sterol ester hydrolase, elastase, kininogenase, ribonuclease, deoxyribonuclease, α-amylase, papain, chymopapain, glutenase, bromelain, ficin, β-amylase, cellulase, β-galactosidase, lactase, sucrase, isomaltase, and mixtures thereof.

The term “pancreatic enzyme” as used herein refers to any one of the enzyme types present in the pancreatic secretion, such as amylase, lipase, protease, or mixtures thereof, or any extractive of pancreatic origin having enzymatic activity, such as pancreatin.

The terms “pancrelipase” or “pancrelipase enzymes” or “pancreatin” denotes a mixture of several types of enzymes, including amylase, lipase, and protease enzymes. Pancrelipase is commercially available, for example from Nordmark Arzneimittel GmbH, or Scientific Protein Laboratories LLC.

The term “lipase” denotes an enzyme that catalyzes the hydrolysis of lipids to glycerol and simple fatty acids. Examples of lipases suitable for the present invention include, but are not limited to animal lipase (e.g., porcine lipase), bacterial lipase (e.g., Pseudomonas lipase and/or Burkholderia lipase), fungal lipase, plant lipase, recombinant lipase (e.g., produced via recombinant DNA technology by a suitable host cell, selected from any one of bacteria, yeast, fungi, plant, insect or mammalian host cells in culture, or recombinant lipases which include an amino acid sequence that is homologous or substantially identical to a naturally occurring sequence, lipases encoded by a nucleic acid that is homologous or substantially identical to a naturally occurring lipase-encoding nucleic acid, etc.), synthetic lipase, chemically-modified lipase, and mixtures thereof. The term “lipids” broadly includes naturally occurring molecules including fats, waxes, sterols, fat-soluble vitamins (such as vitamins A, D, E and K), monoglycerides, diglycerides, triglycerides, phospholipids, etc.

The term “amylase” refers to glycoside hydrolase enzymes that break down starch, for example alfa-amylases, beta-amylases, gamma-amylases, acid α-glucosidases, salivary amylases such as ptyalin, etc. Amylases suitable for use in the present invention include, but are not limited to animal amylases, bacterial amylases, fungal amylases (e.g., Aspergillus amylase, for example, Aspergillus oryzae amylase), plant amylases, recombinant amylases (e.g., produced via recombinant DNA technology by a suitable host cell, selected from any one of bacteria, yeast, fungi, plant, insect or mammalian host cells in culture, or recombinant amylases which include an amino acid sequence that is homologous or substantially identical to a naturally occurring sequence, amylases encoded by a nucleic acid that is homologous or substantially identical to a naturally occurring amylase-encoding nucleic acid, etc.), chemically modified amylases, and mixtures thereof.

The term “protease” refers generally to enzymes (e.g., proteinases, peptidases, or proteolytic enzymes) that break peptide bonds between amino acids of proteins. Proteases are generally identified by their catalytic type, e.g., aspartic acid peptidases, cysteine (thiol) peptidases, metallopeptidases, serine peptidases, threonine peptidases, alkaline or semi-alkaline proteases, neutral and peptidases of unknown catalytic mechanism. Non-limiting examples of proteases suitable for use in the present invention include serine proteases, threonine proteases, cysteine proteases, aspartic acid proteases (e.g., plasmepsin) metalloproteases and glutamic acid proteases. In addition, proteases suitable for use in the present invention include, but are not limited to animal proteases, bacterial proteases, fungal proteases (e.g., an Aspergillus melleus protease), plant proteases, recombinant proteases (e.g., produced via recombinant DNA technology by a suitable host cell, selected from any one of bacteria, yeast, fungi, plant, insect or mammalian host cells in culture, or recombinant proteases, which include an amino acid sequence that is homologous or substantially identical to a naturally occurring sequence, proteases encoded by a nucleic acid that is homologous or substantially identical to a naturally occurring protease-encoding nucleic acid, etc.), chemically modified proteases, and mixtures thereof.

The pancrelipase enzymes of the present invention can include one or more lipases (i.e., one lipase, or two or more lipases), one or more amylases (i.e., one amylase, or two or more amylases), one or more proteases (i.e., one protease, or two or more proteases), as well as mixtures of these enzymes in different combinations and ratios.

Lipase activities in the compositions useful for the present invention can be from about 650 to about 45,000 IU (USP method), from about 675 to about 825 IU, from about 2,500 to about 28,000 IU (USP method), from about 2,700 to about 3,300 IU, from about 4,500 to about 5,500 IU, from about 9,000 to about 11,000 IU, from about 13,500 to about 16,500 IU, and from about 18,000 to about 22,000 IU, from about 22,500 to about 27,500 IU, from about 36,000 to about 44,000 IU, and all ranges and subranges there between. Amylase activities in the compositions can be from about 1,600 to about 6,575 IU (USP), from about 6,000 to about 225,000 IU, for example from about 6,400 to about 26,300 IU, from about 10,700 to about 43,800 IU, from about 21,500 to about 87,500 IU, from about 32,100 to about 131,300 IU, from about 42,900 to about 175,000 IU, from about 53,600 to about 218,700 IU and all ranges and subranges there between. Protease activities in the compositions can be from about 1,250 to about 3,850 IU (USP), from about 5,000 to about 130,000 IU, for example from about 5,000 to about 15,400 IU, from about 8,400 to about 25,700 IU, from about 16,800 to about 51,300 IU, from about 25,000 to about 77,000 IU, from about 33,500 to about 102,800 IU, from about 41,800 IU to about 128,300 IU and all ranges and subranges there between. The lipase activity can range from about 675 to about 825 IU, the amylase activity from about 1,600 to about 6,575 IU, and the protease activity from about 1,250 to about 3,850 IU (USP). The lipase activity can range from about 2,700 to about 3,300 IU, the amylase activity from about 6,400 to about 26,300 IU, and the protease activity from about 5,000 to about 15,400 IU (USP). Or the lipase activity can range from about 4,500 to about 5,500 IU, the amylase activity from about 10,700 to about 43,800 IU, and the protease activity from about 8,400 to about 25,700 IU (USP). Or the lipase activity can range from about 9,000 to about 11,000 IU, the amylase activity from about 21,500 to about 87,500 IU, and the protease activity from about 16,800 to about 51,300 IU (USP). Or the lipase activity from about 13,500 to about 16,500 IU, the amylase activity from about 32,100 to about 131,300 IU, and the protease activity from about 25,000 to about 77,000 IU (USP). The lipase activity can range from about 18,000 to about 22,000 IU, the amylase activity from about 42,900 to about 175,000 IU, and the protease activity from about 33,500 to about 102,600 IU (USP). The lipase activity can range from about 22,000 to about 27,500 IU, the amylase activity from about 53,600 to about 218,700 IU, and the protease activity from about 41,800 IU to about 128,300 IU (USP). Also the lipase activity can range from about 5,000 PhEur lipase units to about 30,000 PhEur lipase units, it may be about 5,000, or about 10,000, or about 15,000 or about 20,000 or about 30,000, or about 40,000 PhEur lipase units.

In one embodiment of the present invention also single units containing a fraction of the above listed amylase activities can also be analysed with the present process.

In certain embodiments, the ratio of amylase/lipase activities in the compositions can range from about 1 to about 10, such as from about 2.38 to about 8.75 (enzymatic assay is performed according to USP). In yet another embodiment, the ratio of protease/lipase can range from about 1 to about 8, such as from about 1.86 to about 5.13 (enzymatic assay is performed according to USP). In still other embodiments, the ratio of amylase/lipase activities is about 1, about 2, about 3, about 4, about 5, about 6, about 7, about 8, about 9, or about 10.

Aptalis Pharma markets at least some of those enterically coated pancrelipase enzymes beads medications that may be used in the present invention. For example, Aptalis Pharma markets delayed-release capsules for the treatment of exocrine pancreatic insufficiency (EPI) in patients under the designation EUR-1008 and the registered trademark Zenpep®. Each Zenpep® capsule for oral administration contains enteric coated beads (1.8-1.9 mm for 750, 3,000, 5,000 USP units of lipase, 2.2-2.5 mm for 10,000, 15,000, 20,000 and 40,000 USP units of lipase).

Aptalis Pharma's preparations replace missing enzymes, improve digestion and absorption, and meet the standards of the United States Pharmacopeia. The Zenpep® capsules containing the enterically coated pancrelipase enzymes is comprised of hydroxypropyl-methylcellulose and have a water content of about 6% or less, preferably of about 2% or less. The inactive ingredients of the product include croscarmellose sodium, hydrogenated castor oil, colloidal silicon dioxide, microcrystalline cellulose, magnesium stearate, hypromellose phthalate, talc, and triethyl citrate. Every dose of Aptalis Pharma's preparations provides patients and physicians with a consistent amount of the main pancreatic enzymes lipase, protease, and amylase due to their highly stable formulation. Capsules can be opened and the content split to individually titrate the dose. These features allow health-care professionals to fine tune treatment regimens to achieve optimal symptom control with improved dosing precision.

In a particular embodiment of the invention, pancrelipase enzymes are introduced into a nutritional enteral feed formula. This procedure comprises the steps of pouring of a portion of a liquid nutritional composition comprising a mixture of carbohydrates, lipids, proteins, micronutrients, trace elements, fibers and water into a blender in amount sufficient to cover all parts of the blades. This is followed by adding the total amount of the dose of digestive enzyme beads into the blender, and mixing this mixture under suitable conditions until an homogenate is obtained. The final volume of the obtained enzyme-nutritional formula is then adjusted with the remaining part of liquid nutritional composition. This mixing is carried out using for example a standard household blender. Due to the variability in blade sizes, shapes and rotation speeds in home blenders, the optimum blending conditions to achieve complete disintegration of the beads without loss of enzymatic activity requires a continuous mixing at mixing speed of between 12,500 and 18,000 rpm for 1-2 minutes at room temperature. Under these condition the obtained homogenate does not contains intact tablets or fragments of appreciable size. Foaming of the formula that is generated during blending resolves over time.

Alternatively, enterically coated pancrelipase enzymes beads may be disintegrated to ensure the availability of the active enzymes by suspending them in a pharmaceutically acceptable weakly basic solution, the solution is then a added to the liquid nutritional composition comprising a mixture of carbohydrates, lipids, proteins and water, and the resultant mixture is finally mixed to form the pancrelipase predigested nutritional formula. In the process the mixture of beads in basic solution is held for about 20 minutes to about 120 minutes before the mixing with the liquid nutritional composition. The mixture is kept preferably for about 20 minutes to about 45 minutes and it may be stirred before pouring it into the nutritional formula. This mixture may be held at below room temperature, including temperatures below 5° C., such as at 4° C.

The weakly basic solution comprises an alkaline substance, amino acid or mixture thereof. The alkaline substance may be selected from the group consisting of alkali and alkaline earth metal hydroxides, carbonates, bicarbonates, sulphates, phosphates and oxides, tris-(hydroxymethyl)-aminomethane (THAM) and mixture thereof. Said alkaline substance may be selected from the sodium, potassium, calcium or magnesium carbonates, bicarbonates, sulphates, phosphates and mixture thereof. In some embodiments, the alkaline substance is selected from the group consisting of sodium bicarbonate, monobasic sodium phosphate, dibasic sodium phosphate, tribasic sodium phosphate, magnesium carbonate, calcium carbonate, and magnesium oxide, and mixtures thereof. In a particular embodiment, the alkaline substance is sodium bicarbonate. When sodium bicarbonate is used than the concentration ranges from about 0.65 to about 13% weight/volume, such as about 8.4% weight/volume. In other embodiments, the sodium bicarbonate concentration is about 0.65%, about 0.70%, about 0.75%, about 0.80%, about 0.85%, about 0.90%, about 0.95%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.4%, about 8.5%, about 9.0%, about 10.0%, about 10.5%, about 11.0%, about 11.5%, about 12.0%, about 12.5%, or about 13.0% weight/volume, inclusive of all ranges and subranges therebetween. The weakly basic solution is prepared by dissolving the suitable amount of the alkaline substance in suitable volume of aqueous medium. The pH of the solution is from about 7.5 to about 8.5, such as from about 8.1 to about 8.2.

The mixture of enterically coated pancrelipase beads in the pharmaceutically acceptable weakly basic solution is prepared in a short period of time and the volume of weakly basic solution required to have an effective disintegration of the enterically coated pancrelipase enzymes is from about 5 mL to about 25 mL (e.g., about 5, about 12, about 15, about 17, about 20, about 22, or about 25 mL) depending on the lipase USP units to be added. This mixture has much higher lipase stability than a corresponding mixture prepared starting from pancrelipase powder.

Different liquid nutritional composition can be used in the present invention; they are prepared as described herein and comprise the carbohydrates in amounts from about 28 to about 90%, the fats in amount from about 1 to about 55%, the proteins in amount from about 4 to about 32% of total calories, the micronutrients meeting 100% of the RDA for vitamins and minerals. Commercial liquid nutritional compositions, such as, but not limited to, PediaSure® or Ensure® or Pulmocare® from Abbott Laboratories or Fresubin® from Fresenius Kab or other similar products may also be used.

Pancrelipase enzymes should be dosed into the liquid nutritional composition; the dose may be adapted for individual patients based on the clinical symptoms, degree of steatorrhea and fat content of the diet. A dose from about 2,000 to about 4,000 lipase units per gram of fat is recommended as the starting dose when mixed with liquid nutritional composition prior to administration. The dosage forms having the pancrelipase enzymes, such as capsules can be opened and the content can be added into the liquid nutritional composition in single or in multiple doses as prescribed by the health care provider as they are or in form of a mixture with a weakly basic solution.

With the process of the instant invention, pancrelipase predigested nutritional formula has high lipase activity which is calculated as percentage of the units of lipase activity added to the liquid nutritional composition; it is above about 85%, such as about 90% or about 95%. After about 360 minute of storage time at about room temperature the mean lipase activity as percentage of units of lipase added to the liquid nutritional composition is above about 95%.

From the foregoing description and the experimental part, it can be seen that the present invention provides several important advantages. The invention provides a simple and fast process suitable for preparation of enteral formula starting from a beaded medicament; the lipase activity is maintained after addition into the liquid nutritional composition; the obtained predigested nutritional formula does not contain particles such as intact tablets or fragments thereof; the lipase remains stable in the liquid meal for over than six hours and the lipolysis is effectively achieved.

The predigested nutritional formula is suitable for use with infant patients, aged patients, or other patients suffering from EPI, which allows medication to be dispensed carefully and with controlled dosing carefully.

The present invention provides also for a kit which combines the liquid nutritional composition (aqueous mixture of carbohydrates, lipids, proteins) contained in a suitable sealed container and the enzyme dosage form in the form of enterically coated pancrelipase enzymes composition. (e.g., capsules 100). The kit may further comprise a pharmaceutically acceptable weakly basic solution or an alkaline substance for use in preparing a pharmaceutically acceptable weakly basic solution.

Glass bottles containing the dosage form such as a certain number of capsules are suitable. Alternatively, the compositions or dosage forms of the present invention can be packaged as a unit dosage form in “blister packs.” To improve stability of the compositions or dosage forms, they should be stored in a sealed, moisture-proof package. Non-limiting examples of suitable moisture-proof packages include glass jars, plastic jars incorporating moisture barrier resins or coatings, aluminized plastic (e.g., Mylar) packaging, and the like. The phrase “moisture-proof” refers to a package which has a water permeability of less than about 0.5 mg of water per cubic centimeter (cm³) of container volume per year. The containers (e.g., bottles), in which the compositions or dosage forms are stored, can be closed with any suitable closure, especially closures that minimize the ingress of moisture during storage. Packages containing the dosage forms to be dispensed with the liquid nutritional composition can also contain a desiccant (i.e., a substance which absorbs, reacts with, or adsorbs water) capable of reducing the humidity inside the package, for example a desiccant capsule capable of “scavenging” moisture from the atmosphere sealed inside the package (such as molecular sieves, clay, silica gel, activated carbon, and mixtures thereof). In addition, it is common practice when packaging oral pharmaceutical unit doses to add a “plug” of a cellulosic material, such as cotton, into the top of the container to fill the empty space at the top of the container, thereby minimizing movement of the content.

The two kit components (liquid nutritional composition, dosage form with enterically coated pancrelipase beads and optionally the alkaline substance) can be packaged together in one single pack.

This kit may be stored in any suitable package which ensures the stability of the product. The package should minimize the ingress of moisture during transportation and/or storage. For example, the package can be a glass or plastic jar with a threaded or press-fit closure.

The present invention also encompasses a method of administration to pediatric or adult patients of the predigested nutritional formula obtained with the process of the invention, comprising the step of a) transferring the predigested nutritional formula to a dispensing bag; and b) dispensing the predigested nutritional formula from the bag to the patient through an enteral tube. The predigested nutritional formula may be gently agitated before its dispensing. This method permits the easy and precise temporary preparation of the predigested nutritional formula starting from enterically coated pancrelipase enzymes.

### EXAMPLES

All experiments, with the exception of Experiment 2.2 and 4.11, are carried out using enterically coated pancrelipase beads, either pancrelipase minitablets (MT) or microtablets (MCT), which are a blend of pancrelipase raw material and excipients (e.g., croscarmellose sodium, hydrogenated castor oil, colloidal silicon dioxide, microcrystalline cellulose, and magnesium stearate) coated with the enteric polymer hypromellose phthalate (HP55); these MTs and MCTs are contained in HPMC capsules having water content below 6%; they are on the market with the name Zenpep®. The skilled artisan will recognize that alternative enteric polymers and excipients may be used in the enterically coated pancrelipase beads.

The liquid nutritional composition (or liquid meal) used in the following experiments has a content of protein: 6.25 g/100 mL, fat: 4.92 g/100 mL, carbohydrate: 20.2 g/100 mL, 1.5 cal/ml (Ensure Plus®, Abbott, 200 mL bottle, flavor: strawberry). Alternative formulations can be used depending on the specific needs of the patient.

1) Measurement of lipolytic activity is carried out according to the compendia procedure of lipase assay described in the Pancrelipase USP monograph, which is based on the titration, by means of pH-stat method, of the free fatty acids formed from the hydrolysis of esterified fatty acids in the substrate used (olive oil). It is based on the following principle: lipase catalyses the hydrolysis of the triglycerides which leads to the formation of free fatty acids (FFA). The titration of the formed FFA according to time provides for the determination of the enzymatic activity of lipase, which can be expressed in units: 1 U=1 μmole of formed FFA per minute. The reaction occurs by maintaining a steady pH value through an experimental system that provides for the addition of sodium hydroxide (titrant) when the pH value changes compared to a fixed value (pHstat method). The quantity of added titrant according to time corresponds to the quantity of FFA formed by the lipase action on the triglycerides. Provided to work with a suitable quantity of substrate and under experimental conditions where the enzyme is stable, a linear kinetics for the FFA formation according to time can be obtained. The curve slope {added titrant=f (time, minutes)} gives the Lipase enzymatic activity.

2) Measurement of proteolytic activity is carried out according to the compendia procedure described in the Pancrelipase USP monograph.

3) Measurement of amilolytic activity is carried out according to the compendia procedure described in the Pancrelipase USP monograph.

### Experiment 1. Pancrelipase Microtablets Mechanical Disintegration in Liquid Meal

### Experiment 1.1.

The bottle containing the liquid meal (protein: 6.25 g/100 mL, fat: 4.92 g/100 mL, carbohydrate: 20.2 g/100 mL, Ensure Plus®, Abbott:) is shaken and then opened and 200 mL of the content is poured into the Sterilmixer 12 Lab homogenizer, equipped with a 500-mL plastic container and stainless steel asymmetrical blades, mixing speed range: 12,500-18,000 rpm (PBI). An amount of pancrelipase MCT (Zenpep®, 61 Lipase USP units/mg) equivalent to 40,000 Lipase USP units (about 600 mg product≈40 microtablets, eight Zenpep® 5000 capsules) approx. 4,000 Lipase USP units per g fat contained in the liquid meal) is added and the blender is closed, the apparatus is started at mixing speed: position 9 of the Blender=16,500 rpm for 1 minute.

The disintegration of the microtablets is checked after the blending in the following way:

a) Half the content of the blender is filtered through a filter crucible (a 30-mL filter crucible, sintered glass disc porosity 0) in a filtering flask with a conical rubber seal to assess the homogeneity of the liquid meal and the absence of intact microtablets. The filter disc is checked: neither intact tablets nor fragments of appreciable size are detected, hence the product is considered to be disintegrated; moreover the filtrate is homogenous as compared with the untreated liquid meal;

b) The remaining part of unfiltered content of blender is transferred into a 200-mL beaker, the beaker is covered and the homogenate is left at room temperature for 30 min, then the bottom is visually checked and found to be homogeneous. The results are summarized in Table 1.

### Experiment 1.2.

The entire procedure of Experiment 1.1 is repeated using a lower mixing speed rate (15,500 rpm corresponding to position 7 of the apparatus) for 1 minute and for 2 minutes and the disintegration of the microtablets is checked in the same way of Experiment 1, results are reported in Table 1.

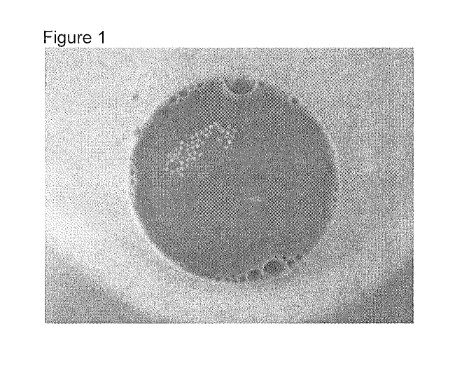

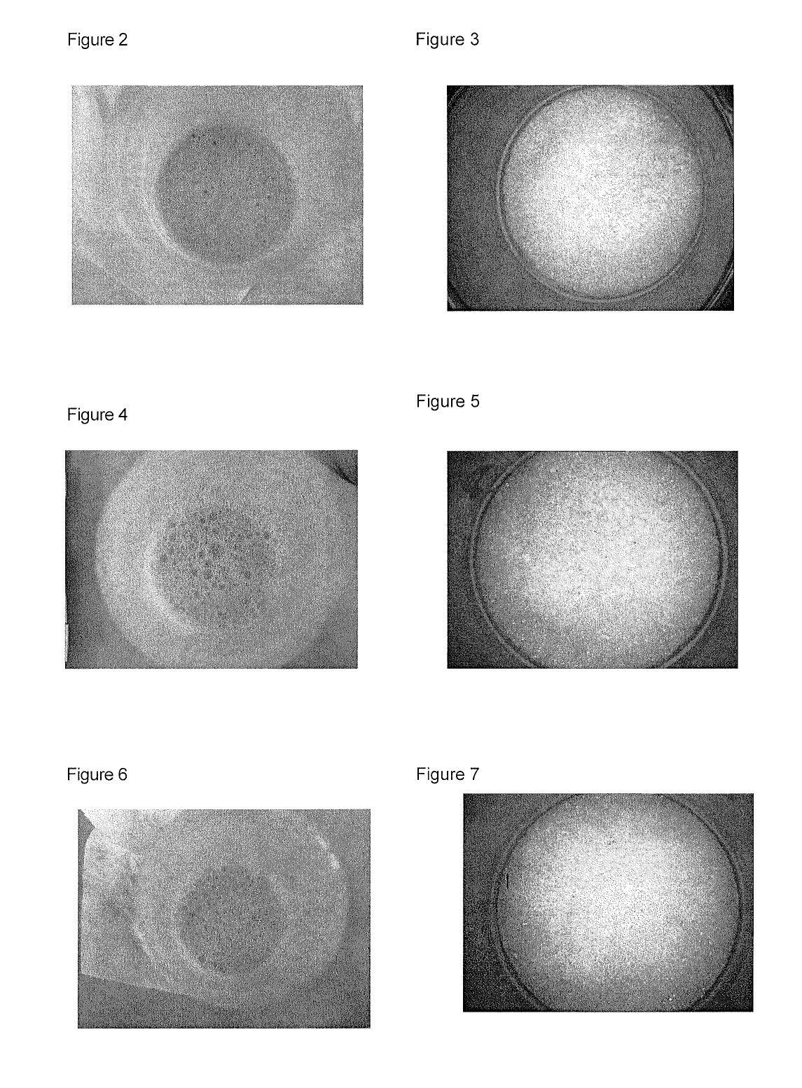

TABLE 1

| | | | | | | Appearance of the |
|---|---|---|---|---|---|---|
| | | Temperature of | Appearance of the liquid | Appearance of the | | unfiltered homogenate |
| Blending | | the homogenate | meal + MCT after | homogenate before | Residue on the | after 30 min storage |
| Time | | after blending | blending | filtration | filter | at r.t. |
| EX. 1.1: Mixing | 1-min | No significant | Homogeneous liquid with | No intact tablets | No intact tablets | Homogenous liquid |
| speed = 16500 rpm | blending | increase | some foam; no changes in | nor fragments | nor fragments of | with few sand-like |
| | | | appearance and aroma with | of appreciable | appreciable size | fragments on the |
| | | | the untreated liquid meal | size observed | (FIG. 3) | bottom of the beaker |
| | | | (FIG. 1) | | | |
| EX 1.2: Mixing | 1-min | No significant | Homogeneous liquid with | No intact tablets | No intact tablets | Homogenous liquid |
| speed = 15500 rpm | blending | increase | some foam; no changes in | nor fragments | nor fragments of | with few sand-like |
| | | | appearance and aroma with | of appreciable | appreciable size | fragments on the |
| | | | the untreated liquid meal | size observed | (FIG. 5) | bottom of the beaker |
| | | | (FIG. 4) | | | |
| | 2-min | No significant | Homogeneous liquid with | No intact tablets | No intact tablets | Homogenous liquid |
| | blending | increase | some foam; no changes in | nor fragments | nor fragments of | with few sand-like |
| | | | appearance and aroma with | of appreciable | appreciable size | fragments on the |
| | | | the untreated liquid meal | size observed | (FIG. 7) | bottom of the beaker |
| | | | (FIG. 6) | | | |

A blending period of 1-2 minutes using the rotary blender is an effective means to homogenously disperse the pancreatic enzymes present in the microtablets in a liquid meal. The in vitro experiment shows that a complete disintegration of the microtablets in the liquid meal is achieved by blending at 15,000 rpm (speed available in home blender) for 1 minute.

Experiment 2. Determination of Lipase Stability and Release in an Enzyme-Nutritional Formula Prepared with Pancrelipase Beads (Enzyme-Nutritional Formula/Pancrelipase Beads) and Mixture of Liquid Nutritional Composition and Pancrelipase Powder (Liquid Nutritional Composition/Pancrelipase Powder).

An amount of microtablets equivalent to 40,000 lipase USP units (8 Zenpep® 5000 capsules) is added to 200 mL of liquid meal to get about 4000 lipase USP units/g fat, and blended for 1 minute at 15,500 rpm mixing speed, as described Experiment 1.1 and 1.2. The lipase activity is determined in the enzyme-nutritional formula/pancrelipase beads over 360 min storage at room temperature.

A second sample is prepared in which pancrelipase powder (88 Lipase USP units/mg; same pancrelipase batch contained in the pancrelipase microtablets, Zenpep®) is added to the liquid nutritional composition at the same dose and hand-shaken for a minute. The comparison with the powder is performed in order to detect any loss of lipase activity in pancrelipase microtablets as a result of the blending procedure.

### Experiment 2.1. Lipase Activity in Enzyme-Nutritional Formula/Pancrelipase

The bottle of liquid meal is shaken, opened and 200 mL of the content is poured into the Sterilmixer 12 Lab blender, equipped with a 500-mL plastic container and stainless steel asymmetrical blades. An amount of microtablets equivalent to 40,000 Lipase USP units (approx. 4,000 Lipase USP units per g fat contained in the liquid meal) is added to the blender, the blender is closed and the apparatus is operated for 1 minute at mixing speed: position 7 of the blender corresponding to 15,500 rpm. The complete disintegration of the microtablets is confirmed by visual check. Immediately after blending a 3-mL aliquot of homogenate is transferred into a 50-mL volumetric flask and dilute to volume with cold purified water; the solution (theoretical lipase concentration is 12 USP units/ml) is shaken briefly. This is the T0 sample. 1 mL of the T0 sample is immediately titrated following the Lipase Assay Pancrelipase USP monograph to determine the lipase activity. The percentage of lipase activity in relation to the theoretical total lipase activity introduced into the liquid meal is determined.

The container is closed and the homogenate is left at room temperature without stirring; before taking each aliquot the homogenate is briefly shaken. The same procedure is repeated after 10, 20, 30, 40, 60, 120, 240, 360 minutes, by taking at each time point fresh 3-mL aliquots of the homogenate stored at room temperature and following the dilution and analysis described for the T0 sample.

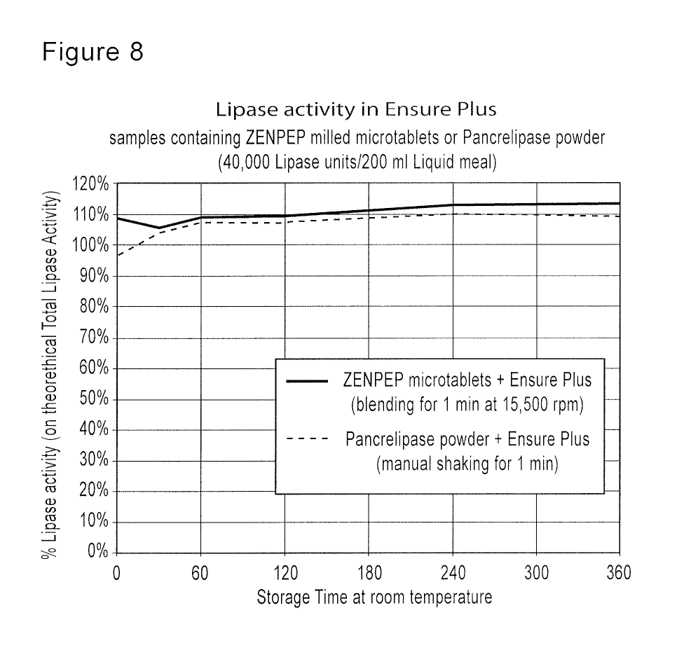

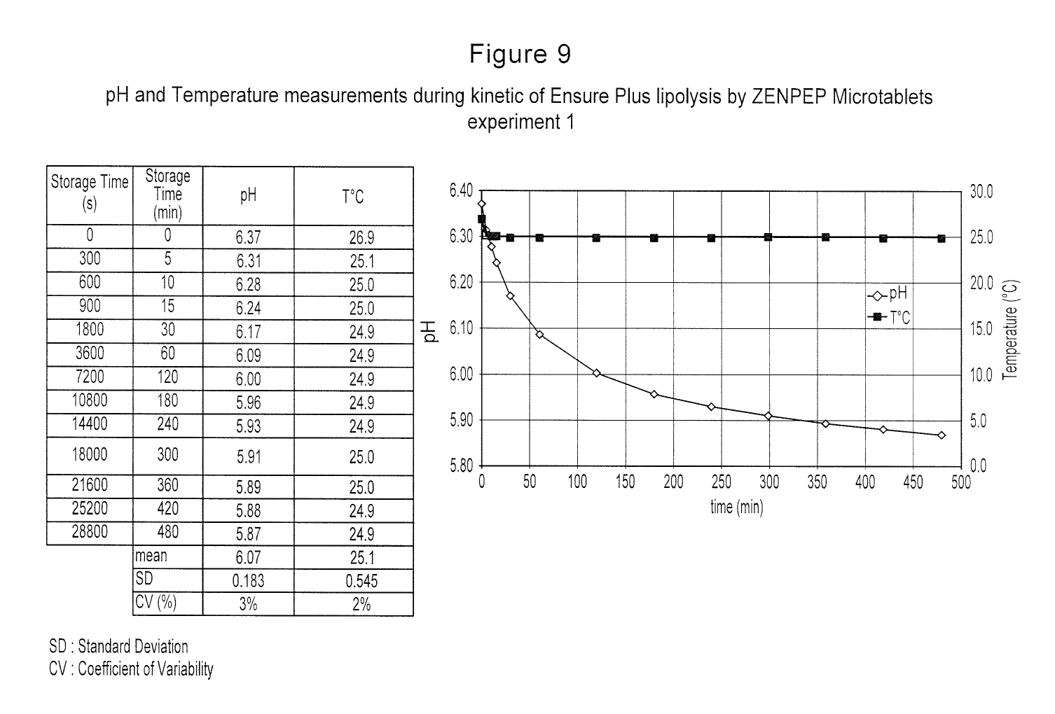

The whole experiment is repeated twice in two different days (replicate 1 and 2) and results are presented in Table 2, see also FIG. 8.

TABLE 2
Lipase activity over time in the enzyme-nutritional formula/pancrelipase beads

| Storage time | Replicate 1 | | Replicate 2 | | Mean lipase |
|---|---|---|---|---|---|
| (min) after 1-min | Total lipase | Lipase activity as | Total lipase | Lipase activity | activity as % |
| blending at | activity | % of theoretical | activity | as % of | of theoretical |
| 15,500 rpm | (USP units) | total¹ | (USP units) | theoretical total² | total |
| Initial | 44513 | 111.3% | 42564 | 106.3% | 108.8% |
| 30 | 43159 | 107.9% | 41503 | 103.6% | 105.8% |
| 60 | 44187 | 110.5% | 42841 | 107.0% | 108.7% |
| 120 | 44563 | 111.4% | 42887 | 107.1% | 109.3% |
| 240 | 46268 | 115.7% | 44018 | 109.9% | 112.8% |
| 360 | 46394 | 116.0% | 44479 | 111.0% | 113.5% |

¹amount of microtablets added to 200 mL liquid meal (655.54 mg) × Batch Lipase assay (61 Lipase USP units/mg) = 39988 USP units;
²amount of microtablets added to 200 mL liquid meal (656.53 mg) × Batch Lipase assay (61 Lipase USP units/mg) = 40048 USP units

### Experiment 2.2. Lipase Activity in Liquid Nutritional Composition/Pancrelipase Powder

The bottle of liquid meal is shaken, opened and then 200 mL of the content is poured into the 500-mL plastic container (same type used for the blender). An amount of pancrelipase powder (same lot of raw material contained in the MCT used for the Experiment 2.1) equivalent to 40,000 Lipase USP units, approx. 4,000 Lipase USP units per g fat contained in the liquid meal) is added and the container is closed and hand-shake well for 1 minute. A 3-mL aliquot of liquid nutritional composition/pancrelipase powder is transferred into a 50-mL volumetric flask and diluted to volume with cold purified water; the solution (theoretical lipase concentration=12 USP units/mL) is briefly shaken to homogenize it. This is the T0 sample. 1 mL of the T0 sample is immediately titrated following the Lipase Assay Pancrelipase USP monograph to determine the lipase activity. The percentage of lipase activity in relation to the theoretical total lipase activity introduced into the liquid meal is determined.

The same procedure is repeated after 10, 20, 30, 40, 60, 120, 240, and 360 minutes, by taking at each point fresh 3-mL aliquots of the liquid nutritional composition/pancrelipase powder stored at room temperature and following the dilution and analysis described for the T0 sample.

The whole experiment is repeated twice in two different days (replicate 1 and 2) and results are presented in Table 3, see also FIG. 8.

TABLE 3
Lipase activity over time in the liquid nutritional composition/pancrelipase powder

| Storage Time | Replicate 1 | | Replicate 2 | | |
|---|---|---|---|---|---|
| (min) | | Lipase activity | | Lipase activity | Mean Lipase |
| after mixing | Total Lipase | as % of | Total Lipase | as % of | activity as % |
| by 1-min | Activity | theoretical | Activity | theoretical | of theoretical |
| shaking | (USP units) | total¹ | (USP units) | total² | total |
| Initial | 40538 | 102.3% | 36024 | 90.8% | 96.6% |
| 30 | 42568 | 107.4% | 39823 | 100.4% | 103.9% |
| 60 | 43869 | 110.7% | 40999 | 103.4% | 107.0% |
| 120 | 43791 | 110.5% | 41125 | 103.7% | 107.1% |
| 240 | 45014 | 113.6% | 42280 | 106.6% | 110.1% |
| 360 | 44363 | 111.9% | 42154 | 106.3% | 109.1% |

¹amount of pancrelipase powder added to 200 mL liquid meal (450.32 mg) × Batch Lipase assay (88 Lipase USP units/mg) = 39628 USP units;
²amount of pancrelipase powder added to 200 mL liquid meal (450.63 mg) × Batch Lipase assay (88 Lipase USP units/mg) = 39655 USP units

From the above experiments it is clear that in the enzyme-nutritional composition/pancrelipase MCT the lipase activity is always at the theoretical value, i.e. the units of lipase added to the liquid meal as microtablets. This demonstrates that the release of the enzyme is complete immediately after the blending step and lipase remains stable in the liquid meal for at least six hours. Moreover, these results support that there is no enzyme degradation occurring during the blending step, and the release of the enzyme from the disintegrated microtablets is complete.

### Experiment 3. Efficacy of Lipase Activity in Liquid Meal Formula Pre-Digested by Pancrelipase MCT (Disintegrated by Blending)

The efficacy of lipase activity on the pre-digested meal obtained after blending of liquid nutritional composition with MCT is performed by measuring the kinetics of lipolysis of the esterified fatty acids (TAG=triglycerides; DAG=diglycerides; MAG=monoglycerides), contained in the liquid meal formula, to free fatty acids (FFA). The experiment followed the general lines below:

a) The amount of lipase in the liquid meal formula is 2800 USP U/g fat, on the basis of maximum daily dosage.

b) The liquid meal formula with composition as described above is used, with pH measurement at the beginning and as a function of time (experiments to be performed at 25° C. in a 50-mL thermostated vessel equipped with a pH electrode).

c) The same batch of pancrelipase microtablets of Experiments 1 and 2 is used.

d) Determination of kinetic profile of lipolysis products, over 8 hours, with time points at 0 (before adding microtablets), 5, 10, 15, 30, 60, 120, 180, 240, 300, 360, 420, and 480 minutes: Lipid extraction and analysis of TAG, DAG, MAG and FFA is carried out by TLC-FID analytical method.

e) Determination of lipase stability profile over the same period of time chosen for the lipolysis kinetics by means of assay of pancreatic lipase activity at the same time points using the assay described in the Pancrelipase USP monograph.

### Experiment 3.1 Kinetics Lipolysis Reaction.

The bottle of liquid meal is shaken and 200 mL of the contents are poured into the blender (Waring commercial laboratory blender 8010E model 38BL40). An amount of pancrelipase microtablets (61 lipase USP Units/mg) equivalent to 28,000 lipase USP units is added=approx. 2,800 lipase USP Units per gram of fat contained in the liquid meal. After the 1-minute blending (mixing speed: 18,000 rpm) the complete disintegration of the microtablets is confirmed by visual check. 50 mL of the mixture are transferred into a 50-mL thermostated vessel equipped with a pH electrode. The lipolysis reaction is kept at 25° C. for 480 minutes under no-stirring conditions; the pH and temperature of the reaction are measured at each sampling time point. The initial amount of lipids present (total lipid extraction and TLC-FID analyses) in the liquid meal is measured “as is”, before adding the microtablets (time 0 sample).

### Experiment 3.2. Sampling and Lipid Extraction.

At each time point of the experiment (5, 10, 15, 30, 60, 120, 180, 240, 300, 360, 420, 480 minutes) 1 mL of the reaction mixture is extracted to quantitatively recover the lipolysis products. Before each sampling, the reaction mix is briefly homogenized with mechanical stirrer. The extraction is performed according to Folch's procedure.

### Experiment 3.3. Quantitative Analysis of Lipolysis Products.

The quantitative analysis of TAG, DAG, MAG and FFA is performed by thin layer chromatography technique coupled with a Flame Ionization Detector for the analyte detection. Standard compounds for each lipolysis product (Triolein for TAG; 1,2-diolein for DAG; 1-monoolein for MAG; oleic acid for FFA) are used for the calibration. The global extraction yield is evaluated by calculating the recovery rate of a suitable Internal Standard in the extracted organic layer, by using the corresponding calibration curve. All analyses are performed in duplicate.

### Experiment 3.4. Lipolysis Level Calculation.

Upon hydrolysis, one molecule of TAG can release a maximum of 3 molecules of FFA. The hydrolysis (or lipolysis) level is usually defined as the percentage of acyl chains released from the meal triglycerides (TAG0):

L%=100×FFA/3×TAG₀

The complete absorption of fat requires only the conversion of meal TAG into MAG, which corresponds to the release of two FFA from one TAG molecule, i.e. a 66.6% level of lipolysis according to the above definition. Herein, a definition of the lipolysis level reflecting directly the fat absorption capacity during the enzymatic hydrolysis process is used. The lipolysis level is expressed here as the percentage of the total meal TAG acyl chains converted into “intestinally absorbable” acyl chains, i.e. FFA and MAG. It is defined by the following equation, in which TAG, DAG, MAG and FFA are the amounts in mmoles of residual triglycerides and lipolysis products recovered at a given time during the hydrolysis process:

L%=100×(FFA+MAG)/3×TAG₀=100×(FFA+MAG)/3TAG+2DAG+MAG+FFA

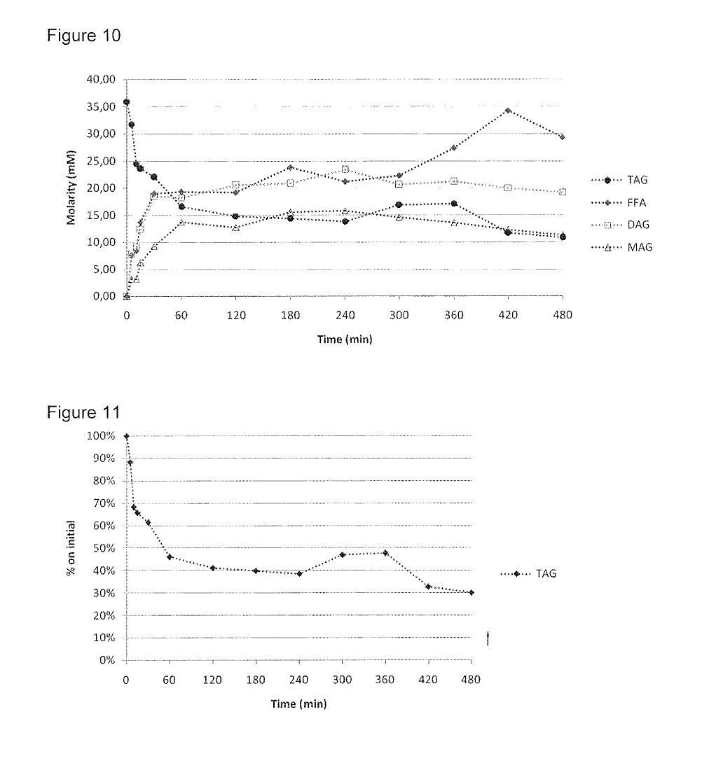

According to this definition, 100% lipolysis corresponds to the conversion of one TAG molecule into one MAG and two FFA molecules. This definition of the level of lipolysis does not take the possible hydrolysis of MAGs into account, the latter process being not essential to fat absorption. The results of the analysis are reported in the following Tables 4-7 and FIGS. 10-12.

TABLE 4
Kinetics of liquid meal lipolysis by pancrelipase
MCT, experiment 3-assay 1

| | |
|---|---|
| sampling interval | Molarity (mM) |

| (min) | TAG | FFA | DAG | MAG |
|---|---|---|---|---|
| 0 | 35.56 | 0.00 | 0.00 | 0.00 |
| 5 | 31.40 | 6.74 | 7.90 | 2.69 |
| 10 | 22.20 | 8.10 | 8.50 | 2.79 |
| 15 | 23.05 | 12.90 | 12.56 | 5.93 |
| 30 | 23.86 | 16.64 | 20.22 | 10.31 |
| 60 | 17.19 | 17.90 | 19.42 | 13.29 |
| 120 | 13.96 | 23.50 | 20.02 | 11.83 |
| 180 | 14.84 | 22.40 | 22.53 | 15.97 |
| 240 | 13.76 | 22.15 | 22.66 | 14.99 |
| 300 | 16.17 | 18.39 | 20.64 | 13.79 |
| 360 | 17.44 | 29.01 | 21.08 | 13.84 |
| 420 | 11.61 | 35.61 | 19.99 | 12.57 |
| 480 | 10.61 | 29.94 | 19.31 | 11.39 |

TABLE 5
Kinetics of liquid meal lipolysis by pancrelipase
MCT, experiment 3-assay 2

| | |
|---|---|
| Sampling interval | Molarity (mM) |

| (min) | TAG | FFA | DAG | MAG |
|---|---|---|---|---|
| 0 | 36.30 | 0.00 | 0.00 | 0.00 |
| 5 | 32.09 | 8.53 | 7.53 | 3.65 |
| 10 | 26.82 | 8.75 | 9.56 | 3.58 |
| 15 | 24.03 | 14.40 | 12.25 | 6.41 |
| 30 | 20.20 | 21.25 | 16.91 | 8.07 |
| 60 | 15.86 | 20.64 | 17.04 | 14.10 |
| 120 | 15.63 | 14.90 | 20.95 | 13.60 |
| 180 | 13.82 | 25.31 | 19.06 | 15.04 |
| 240 | 13.90 | 20.14 | 24.03 | 16.66 |
| 300 | 17.61 | 26.20 | 20.52 | 15.37 |
| 360 | 16.78 | 25.64 | 21.21 | 13.27 |
| 420 | 11.90 | 32.95 | 20.02 | 11.91 |
| 480 | 10.98 | 28.76 | 19.07 | 11.09 |

TABLE 6
Kinetics of liquid meal lipolysis by pancrelipase
Microtablets - experiment 3-Mean

| | |
|---|---|
| Sampling interval | Molarity (mM) |

| (min) | TAG | FFA | DAG | MAG |
|---|---|---|---|---|
| 0 | 35.93 | 0.00 | 0.00 | 0.00 |
| 5 | 31.75 | 7.64 | 7.72 | 3.17 |
| 10 | 24.51 | 8.43 | 9.03 | 3.19 |
| 15 | 23.54 | 13.65 | 12.41 | 6.17 |
| 30 | 22.03 | 18.95 | 18.57 | 9.19 |
| 60 | 16.53 | 19.27 | 18.23 | 13.70 |
| 120 | 14.80 | 19.20 | 20.49 | 12.72 |
| 180 | 14.33 | 23.86 | 20.80 | 15.51 |
| 240 | 13.83 | 21.15 | 23.35 | 15.83 |
| 300 | 16.89 | 22.30 | 20.58 | 14.58 |
| 360 | 17.11 | 27.33 | 21.15 | 13.56 |
| 420 | 11.76 | 34.28 | 20.01 | 12.24 |
| 480 | 10.80 | 29.35 | 19.19 | 11.24 |

TABLE 7
Lipolysis level 1 and 2 according to following calculation:
Lipolysis level 1 calculation (FFA %)
L % = (100 × FFA)/(3 × TAG₀) = (100 × FFA)/(3 ×
TAG + 2 × DAG + MAG + FFA)
Lipolysis level 2 calculation (FFA % = MAG %).
L % = (100 × FFA + MAG)/(3 × TAG₀) = (100 ×
FFA + MAG)/(3 × TAG + 2 × DAG + MAG + FFA).
Lipolysis Levels

| | | Lipolysis | | | Total |
|---|---|---|---|---|---|
| | Lipolysis | level 2 | IS- | | amount of |
| | level 1 | (FFA + | recovery | Time | fatty acids |
| Sample ID | (FFA %) | MAG %) | (%) | (min) | mM |
| Sample 1 assay 1 | 0.0% | 0.0% | 98% | 0 | 107 |
| Sample 1 assay 1 | 5.6% | 7.9% | 90% | 5 | 119 |
| Sample 1 assay 1 | 8.6% | 11.5% | 94% | 10 | 95 |
| Sample 1 assay 1 | 11.4% | 16.7% | 86% | 15 | 113 |
| Sample 1 assay 1 | 12.0% | 19.4% | 94% | 30 | 139 |
| Sample 1 assay 1 | 14.7% | 25.6% | 94% | 60 | 122 |
| Sample 1 assay 1 | 20.0% | 30.1% | 98% | 120 | 117 |
| Sample 1 assay 1 | 17.5% | 30.0% | 86% | 180 | 128 |
| Sample 1 assay 1 | 17.9% | 30.0% | 98% | 240 | 124 |
| Sample 1 assay 1 | 15.1% | 26.4% | 108% | 300 | 122 |
| Sample 1 assay 1 | 21.1% | 31.2% | 91% | 360 | 137 |
| Sample 1 assay 1 | 29.0% | 39.2% | 102% | 420 | 123 |
| Sample 1 assay 1 | 26.8% | 37.0% | 98% | 480 | 112 |
| | | | | mean | 120 |
| | | | | SD | 12 |
| | | | | CV | 10% |
| | | | | (%) | |
| Sample 1 assay 2 | 0.0% | 0.0% | 100% | 0 | 109 |
| Sample 1 assay 2 | 6.9% | 9.9% | 90% | 5 | 124 |
| Sample 1 assay 2 | 7.8% | 11.0% | 82% | 10 | 112 |
| Sample 1 assay 2 | 12.3% | 17.7% | 89% | 15 | 117 |
| Sample 1 assay 2 | 17.2% | 23.7% | 104% | 30 | 124 |
| Sample 1 assay 2 | 17.7% | 29.8% | 96% | 60 | 116 |
| Sample 1 assay 2 | 12.7% | 24.3% | 101% | 120 | 117 |
| Sample 1 assay 2 | 21.1% | 33.6% | 108% | 180 | 120 |
| Sample 1 assay 2 | 15.9% | 29.1% | 108% | 240 | 127 |
| Sample 1 assay 2 | 19.3% | 30.7% | 102% | 300 | 135 |
| Sample 1 assay 2 | 19.5% | 29.5% | 99% | 360 | 132 |
| Sample 1 assay 2 | 27.3% | 37.2% | 107% | 420 | 121 |
| Sample 1 assay 2 | 25.9% | 35.9% | 113% | 480 | 111 |
| | | | | mean | 120 |
| | | | | SD | 8 |
| | | | | CV | 7% |
| | | | | (%) | |

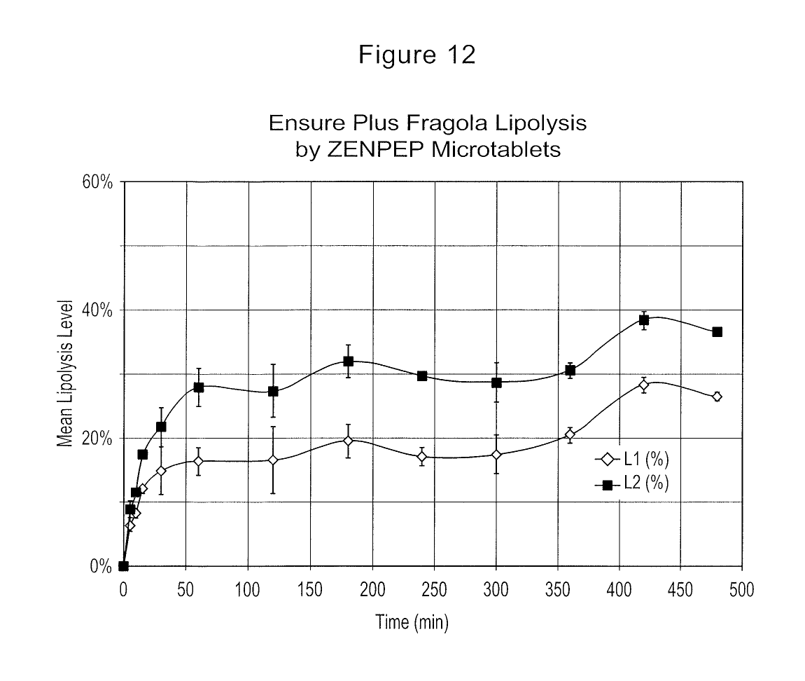

TABLE 8
Mean lipolysis level 1 and 2 according to calculation shown
in Table 7; the value are reported in graph in FIG. 12
Mean Lipolysis Levels

| Time | Lipolysis level 1 (FFA %) | | | |
|---|---|---|---|---|
| (min) | Assay 1 | Assay 2 | mean | SD |
| 0 | 0.0% | 0.0% | 0.0% | 0% |
| 5 | 5.6% | 6.9% | 6% | 1% |
| 10 | 8.6% | 7.8% | 8% | 1% |
| 15 | 11.4% | 12.3% | 12% | 1% |
| 30 | 12.0% | 17.2% | 15% | 4% |
| 60 | 14.7% | 17.7% | 16% | 2% |
| 120 | 20.0% | 12.7% | 16% | 5% |
| 180 | 17.5% | 21.1% | 19% | 3% |
| 240 | 17.9% | 15.9% | 17% | 1% |
| 300 | 15.1% | 19.3% | 17% | 3% |
| 360 | 21.1% | 19.5% | 20% | 1% |
| 420 | 29.0% | 27.3% | 28% | 1% |
| 480 | 26.8% | 25.9% | 26% | 1% |
| Time | Lipolysis level 2 (FFA + MAG %) | | | |
| (min) | Assay 1 | Assay 2 | mean | SD |
| 0 | 0.0% | 0.0% | 0% | 0% |
| 5 | 7.9% | 9.9% | 9% | 1% |
| 10 | 11.5% | 11.0% | 11% | 0% |
| 15 | 16.7% | 17.7% | 17% | 1% |
| 30 | 19.4% | 23.7% | 22% | 3% |
| 60 | 25.6% | 29.8% | 28% | 3% |
| 120 | 30.1% | 24.3% | 27% | 4% |
| 180 | 30.0% | 33.6% | 32% | 3% |
| 240 | 30.0% | 29.1% | 30% | 1% |
| 300 | 26.4% | 30.7% | 29% | 3% |
| 360 | 31.2% | 29.5% | 30% | 1% |
| 420 | 39.2% | 37.2% | 38% | 1% |
| 480 | 37.0% | 35.9% | 36% | 1% |

From the above data it is clear that a remarkable reduction of the TAG level (as the result of the lipolysis activity of lipase) in the liquid meal pre-digested with pancrelipase microtablets (disintegrated by blending) is observed. The concentration of TAG dropped to about 45% of the initial value in the first hour, then continued to decrease to a lesser degree in the remaining part of the experiment, reaching about a 70% decrease after 8 hours. Moreover, with regard to lipolysis these results show that the liquid nutritional composition obtained with the process of the invention has a percentage of acyl chains released from the triglycerides of about 16% after 1 hours and about 28% after 8 hours; the percentage of the triglycerides acyl chains converted into free fatty acid acyl chains and into monoglyceride acyl chains is about 28% after 1 hour and about 36% after 8 hours.

### Experiment 4. Disintegration of Pancrelipase Beads (Microtablets and Minitablets) in Sodium Bicarbonate Solutions.

Preparation of Sodium Bicarbonate Solutions.

Solution A) 13% weight/volume sodium bicarbonate solution: 13.0 g sodium bicarbonate is added to 100 mL of water in a volumetric flask and shaken; the salt does not dissolve completely (saturated solution).

Solution B) 8.4% weight/volume sodium bicarbonate solution: 8.4 g sodium bicarbonate is added to 100 a 100 mL of water in volumetric flask and shaken until dissolved.

Solution C) 0.65% weight/volume sodium bicarbonate solution: 0.65 g sodium bicarbonate is added to 100 mL of water in a volumetric flask and shaken until dissolved.

Experiment 4. Disintegration Time of a Single Dose Unit of Enterically Coated Pancrelipase Enzymes Microtablets with 5,000 Lipase USP Units/Capsule (Zenpep® Microtabs 5,000 Lipase USP U/cps) in 5 mL 8.4% Sodium Bicarbonate Solution at Room Temperature.

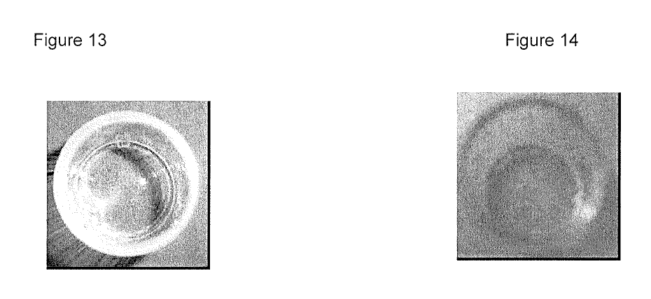

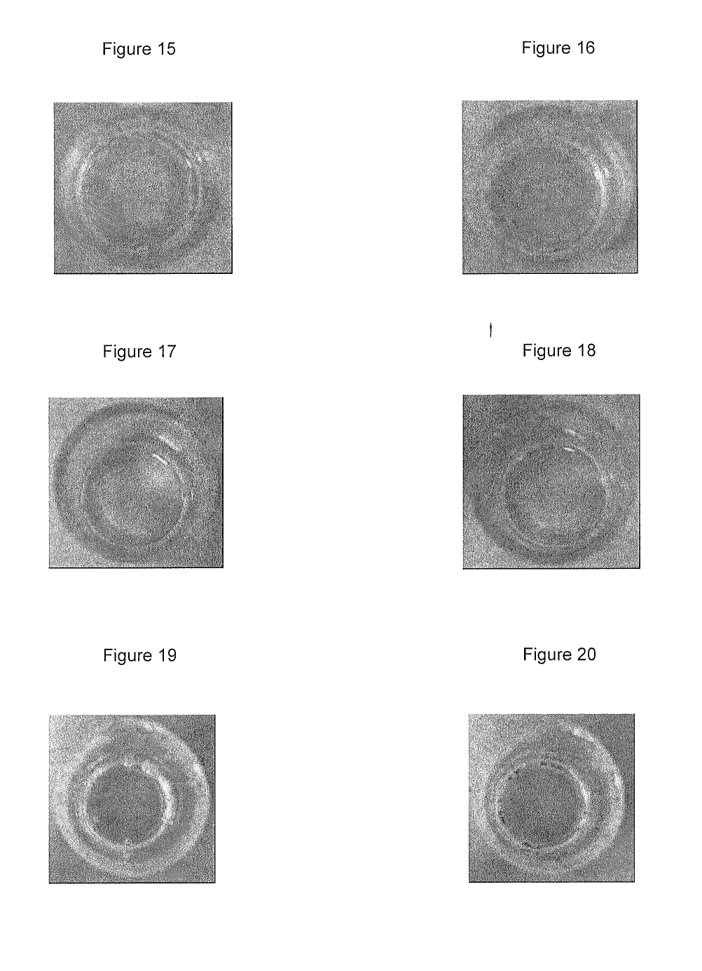

The contents of one capsule are added to 5 mL of 8.4% sodium bicarbonate in a 15 mL beaker, without stirring. The sample is stored at r.t., bench top conditions, without stirring. At regular time intervals visual observations is made, recording the appearance of the soaked microtablets, until a disintegration of the product (that is, any residue of the product is a soft mass having no palpably firm core) is observed. The microtablets appear disintegrated after 20 minute storage in the above conditions. Any remaining residue is completely dissolved after about 55 minutes. FIGS. 13-18 are pictures of the microtablets soaked in the 8.4% bicarbonate solution after 0, 10, 20, 35, 45, and 55 minutes without stirring.

Experiment 4.2. Disintegration of a Single Dose Unit of Pancrelipase Enzymes Microtablets With 5,000 Lipase USP Units/Capsule (Zenpep® Microtabs 5,000 Lipase USP U/cps) after 20 Minutes Storage in 5 mL 8.4% Sodium Bicarbonate Solution at Room Temperature

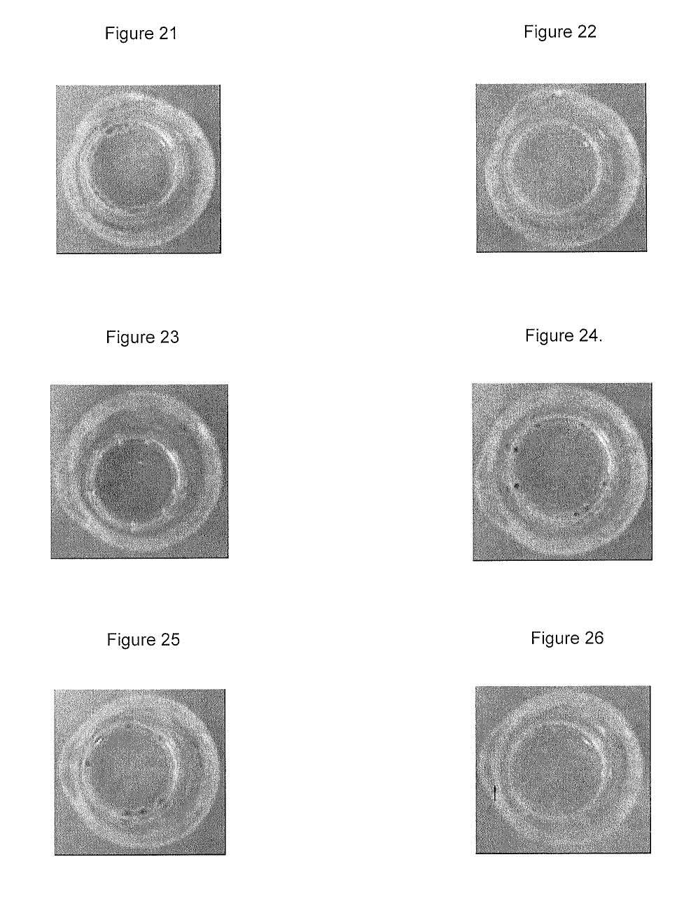

The effectiveness of disintegration of one dose unit is evaluated after 20 min storage in 5 mL 8.4% sodium bicarbonate solution at r.t., after mild stirring of the remaining residues of the product: only a few fragments are observed. FIGS. 19-21 are pictures of the microtablets soaked in the 8.4% bicarbonate solution without stirring after 0, 10, 20 minutes. FIG. 22 is the remaining residues after mild stirring at the end of the 20 min soaking.

Experiment 4.3. Disintegration of Pancrelipase Minitablets in Amount Equivalent to 5,000 Lipase USP Units after 20 Minutes Storage in 5 mL 8.4% Sodium Bicarbonate Solution at Room Temperature.

Experiment 4.2 is repeated using pancrelipase minitablets equivalent to 5,000 Lipase USP U; in this case, in the sample stirred after 20 min soaking in 8.4% sodium bicarbonate solution more fragments are observed.

FIGS. 23-25 are pictures of the minitablets soaked in the 8.4% bicarbonate solution without stirring after 0, 10, and 20 min. FIG. 26 is of the remaining residues after mild stirring at the end of the 20 min soaking.

Experiment 4.4. Disintegration of Pancrelipase Minitablets in Amount Equivalent to 5,000 Lipase USP U after 30 Minutes Storage in 5 mL 8.4% Sodium Bicarbonate Solution at Room Temperature.

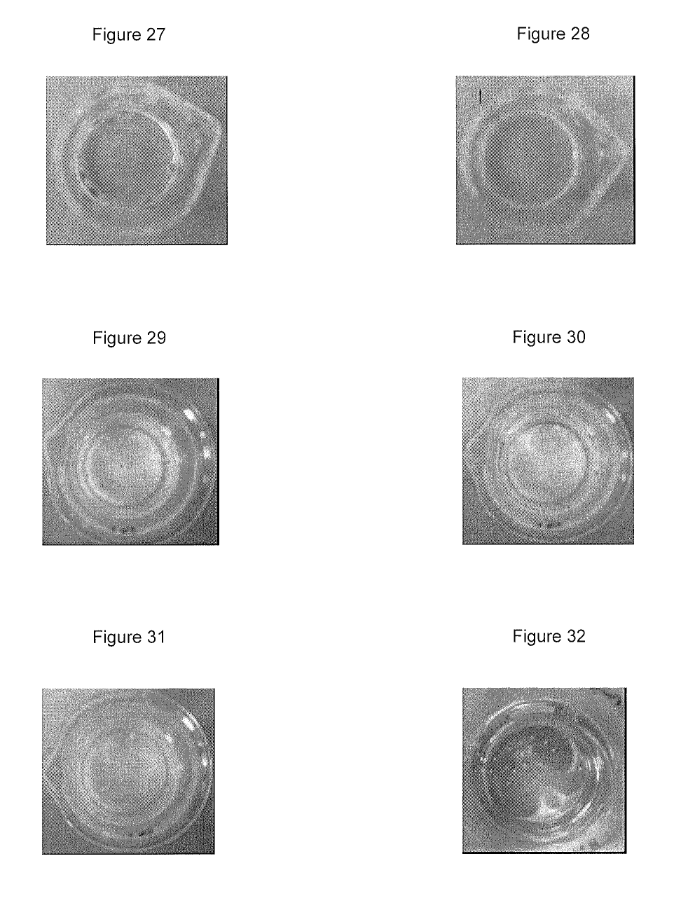

In this experiment, the minitabs in the sodium bicarbonate solution have a longer residence time (30 min) without stirring; at the endpoint the remaining soft masses completely disappeared after a mild stirring (FIGS. 27-28).

Experiment 4.5. pH Measurement of 8.4% Sodium Bicarbonate Solution Added with Pancrelipase Microtablets with 5,000 Lipase USP Units/Capsule (Zenpep® Microtabs 5,000 Lipase USP U/cps) [*Disintegration Stage=0-240 Min].

The pH of 5 mL 8.4% sodium bicarbonate solution at r.t. is measured “as is” and after 5, 10, 15, 20, 30, 80, 120, 180, and 240 min from the addition of the content of a single dose unit. No significant change in pH is observed; Table 9 shows the pH values obtained.

TABLE 9
pH values of a 5 mL 8.4% sodium bicarbonate solution
added with one dose unit pancrelipase microtablets
5,000 lipase USP Units/cps

| Time (min) | pH | |
|---|---|---|
| 0 | 8.223 | Bicarbonate solution “as is” |
| 5 | 8.286 | Bicarbonate solution added with one dose unit of |
| | | pancrelipase microtablets 5,000 lipase USP U/cps |
| 10 | 8.292 | Bicarbonate solution added with one dose unit of |
| | | pancrelipase microtablets 5,000 lipase USP U/cps |
| 15 | 8.298 | Bicarbonate solution added with one dose unit of |
| | | pancrelipase microtablets 5,000 lipase USP U/cps |
| 20 | 8.307 | Bicarbonate solution added with one dose unit of |
| | | pancrelipase microtablets 5,000 lipase USP U/cps |
| 30 | 8.315 | Bicarbonate solution added with one dose unit of |
| | | pancrelipase microtablets 5,000 lipase USP U/cps |
| 80 | 8.352 | Bicarbonate solution added with one dose unit of |
| | | pancrelipase microtablets 5,000 lipase USP U/cps |
| 120 | 8.378 | Bicarbonate solution added with one dose unit of |
| | | pancrelipase microtablets 5,000 lipase USP U/cps |
| 180 | 8.404 | Bicarbonate solution added with one dose unit of |
| | | pancrelipase microtablets 5,000 lipase USP U/cps |
| 240 | 8.410 | Bicarbonate solution added with one dose unit of |
| | | pancrelipase microtablets 5,000 lipase USP U/cps |

Experiment 4.6. Disintegration of a Single Dose Unit of Pancrelipase Enzymes Microtablets with 5,000 Lipase USP U/Capsule (Zenpep® Microtabs 5,000 Lipase USP U/cps) in 5 mL 13% Sodium Bicarbonate Solution at Room Temperature.

The contents of one capsule are added to 5 mL of 13% sodium bicarbonate in a 15 mL beaker, without stirring. The sample is stored at r.t., bench top conditions, without stirring. At regular time intervals visual observations are made, recording the appearance of the soaked microtablets, until disintegration of the product is observed. The microtablets completely dissolve after 20 min storage in the above conditions. FIGS. 29-31 are pictures of the microtablets soaked in 13% bicarbonate solution after 0, 10, and 20 min without stirring.

Experiment 4.7. Disintegration of a Single Dose Unit of Pancrelipase Enzymes Microtablets with 5,000 Lipase USP U/Capsule (Zenpep® Microtabs 5,000 Lipase USP U/cps) in 5 mL 0.65% Sodium Bicarbonate Solution at Room Temperature.

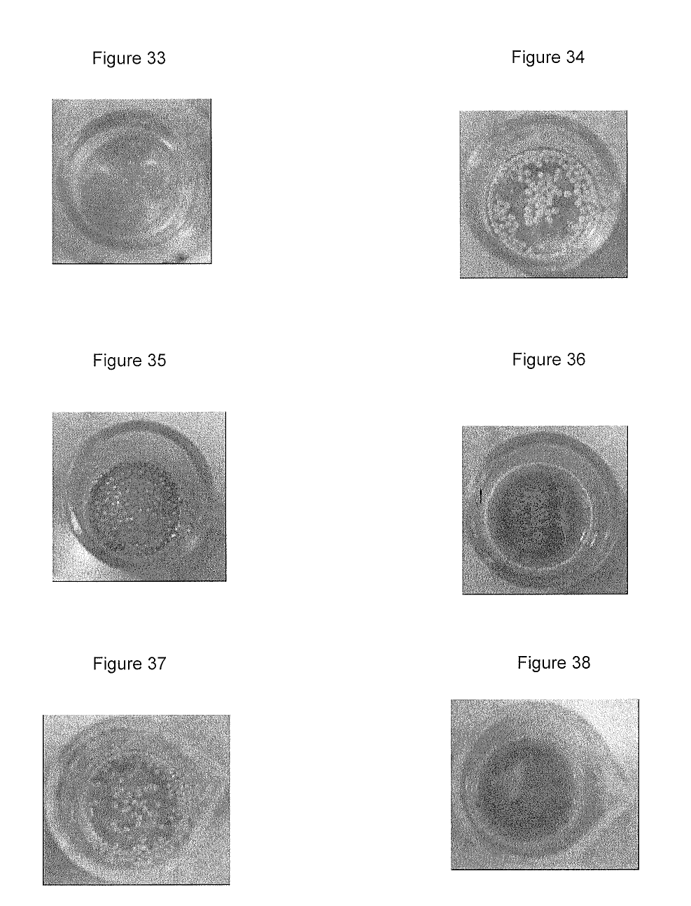

The contents of one capsule are added to 5 mL of 0.65% sodium bicarbonate in a 15 mL beaker, without stirring. The sample is stored at r.t., bench top conditions, without stirring. At regular time intervals visual observations are made, recording the appearance of the soaked microtablets, until a disintegration of the product is observed. After 20 min under the above conditions, all of the microtablets are not completely disintegrated (small hard core still visible). After 35 min only a few soft masses are observed. FIGS. 32-33 are pictures of the microtablets soaked in the 0.65% bicarbonate solution after 0 and 35 min without stirring.

Experiment 4.8. Disintegration of a Pooled Sample of 8 Units of Pancrelipase Enzymes microtablets with 5,000 lipase USP U/Capsule (Zenpep® Microtabs 5,000 Lipase USP U/cps) (5,000 Lipase USP U/Capsule=40,000 Lipase USP U) in 5 mL 8.4% Sodium Bicarbonate Solution at Room Temperature.

The contents of eight capsules with 5,000 Lipase USP U/cps, corresponding to 40,000 lipase USP units, are added to 5 mL of 8.4% sodium bicarbonate in a 30 mL beaker, without stirring. The sample is stored at r.t., bench top conditions, without stirring. At regular time intervals visual observations are made, recording the appearance of the soaked microtablets, until a disintegration of the product is observed. After 20 minutes in the above conditions all the microtablets are not completely disintegrated (a small hard core still visible). After about 40-45 min only soft masses are observed. FIGS. 34-36 are pictures of the microtablets soaked in the 8.4% bicarbonate solution after 0, 20, and 45 min without stirring.

Experiment 4.9. Disintegration of Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) in 5 mL 8.4% Sodium Bicarbonate Solution at 4° C.

The contents of eight capsules corresponding to approximately 40,000 lipase USP units, are added to 5 mL of 8.4% sodium bicarbonate in a 30 mL beaker, without stirring. The sample is stored at 4° C., without stirring. At regular time intervals visual observations are made, recording the appearance of the soaked microtablets, until the disintegration of the product is observed. After 20 min in the above conditions most of microtablets are still intact; after about 45 min only few soft masses are observed. FIGS. 37-38 are pictures of the microtablets soaked in the 8.4% bicarbonate solution at 4° C. after 0 and 45 min without stirring.

Experiment 4.10. Effect of the Volume of 8.4% Sodium Bicarbonate Solution on the Disintegration Time of a Pooled Sample of 8 Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U).

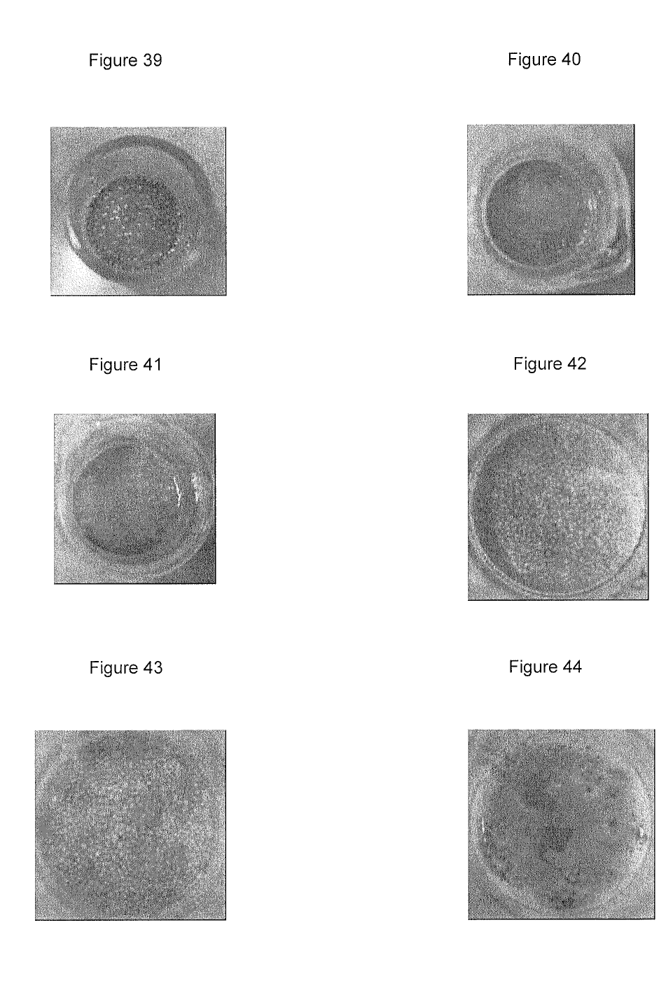

The effect of the volume of 8.4% sodium bicarbonate solution on pancrelipase microtablets (40,000 lipase USP Units) disintegration time is evaluated on the microtablets suspended in 5, 10, 15, 20, and 25 mL of the medium, at r.t., without stirring. At regular time intervals visual observations are made, recording the appearance of the soaked microtablets. A gradual decrease of the microtablets disintegration time with the increasing volume of sodium bicarbonate solution is observed: by increasing the volume from 5 to 10 mL the disintegration time is shortened by about 10 min (35 vs 45 min), the 15 mL sample shows most of the microtablets still intact after 20 min, while in the 20 and 25 mL samples only soft masses are observed after 20 min (same time required for the disintegration of an individual dose unit of 5,000 U in 5 mL of 8.4% bicarbonate solution). The pH of 25 mL sodium bicarbonate solution “as is” and after the 20 min disintegration stage of the microtabs is 8.116 and 7.979, respectively. FIGS. 39-41 are of microtablets soaked in increasing volumes (5, 15, and 25 mL) of 8.4% bicarbonate solution at r.t. after 20 min without stirring.

Experiment 4.11. Disintegration Time of a Pooled Sample of Pancrelipase Enzymes Pellets Corresponding to 40,000 Lipase USP Units (Creon® Delayed Release Capsule, 24,000 Lipase USP U/cps, Product on the Market, Expiry Date 0272012) in 25 mL 8.4% Sodium Bicarbonate Solution at Room Temperature.

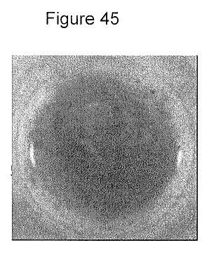

The contents of some capsules corresponding to approximately 40,000 lipase USP units are added to 25 mL of 8.4% sodium bicarbonate in a 50 mL beaker, without stirring. The sample is stored at r.t, without stirring. At regular time intervals visual observations are made, recording the appearance of the soaked microtablets, until the disintegration of the product is observed. After 20 min in the above conditions all granules are still intact, without any significant change in the appearance compared with time 0; after a short stirring, a packed mass of the granules formed. After 60 min a consistent amount of granules having a hard core is still present, which do not disintegrate after another short stirring step. In the further control at 90 min few granules are still observed; the complete disintegration occurs after 120 minutes. FIGS. 42-45 show the pictures of the enteric-coated spheres (Creon®) soaked in the 8.4% bicarbonate solution at r.t. after 0, 20, 90, and 120 min.

### Experiment 5. Determination of Pancrelipase Enzymes Activity in 8.4% Sodium Bicarbonate Solution.

The activity of the three main enzymes contained in pancrelipase enzymes microtablets dissolved in sodium bicarbonate solutions stored at room temperature and 4° C., is evaluated either on a single dose unit of 5,000 lipase USP units and on the higher dose of 40,000 lipase USP units. The activity is ascertained using the methods described above.

Experiment 5.1. Lipase Activity of a Single Dose Unit of Pancrelipase Enzymes Microtablets with 5,000 Lipase USP U/Capsule (Zenpep® Microtabs 5,000 Lipase USP U/cps) Dissolved* in 5 mL 8.4% Sodium Bicarbonate Solution, R.T. vs 4° C. [*Disintegration Stage=20 min at R.T.].

The contents of one capsule are added to 5 mL of 8.4% sodium bicarbonate in a 15 mL beaker, without stirring. The sample is stored at r.t., bench top conditions, without stirring. Two independent samples are prepared (1A and 1B). After 20 min the pancrelipase/bicarbonate samples are stirred and a 120 μl aliquot is diluted to 10 mL water and the lipase activity is determined with 1 mL of this solution, following the Lipase Assay described in the Pancrelipase USP monograph (time 0). Immediately after the t0 sampling sample 1A is stored at room temperature, while sample 1B is kept at 4° C. Further 120 μl aliquots are withdrawn from both samples 1A and 1B after 15, 30, 45, 60, 90, 150, and 240 min from t0, diluted and immediately assayed for the lipase activity. The activity of lipase in the pancrelipase/sodium bicarbonate mixture is expressed as % of the total lipase activity calculated from the batch Lipase Assay found in the same run. The results from two runs are summarized in Table 10a (activity at room temperature), Table 10b (activity at 4° C.) and Table 10c (mean data).

TABLE 10a
Lipase activity of pancrelipase microtabs 5,000 U in 8.4% sodium bicarbonate solution stored at r.t.

| | RUN 1 | | | RUN 2 | | |
|---|---|---|---|---|---|---|
| | Total lipase | % Lipase activity | % Lipase | Total lipase | % Lipase activity | % Lipase |
| Storage time | activity | of theor. total | activity | activity | of theor. total | activity |
| (min) | (USP U) | lipase ² | on t0 | (USP U) | lipase ³ | on t0 |
| Initial (t0) ¹ | 5005 | 96.5 | 100.0 | 4332 | 97.3 | 100.0 |
| 15 | 3917 | 75.5 | 78.3 | 3204 | 72.0 | 74.0 |
| 30 | 2974 | 57.3 | 59.4 | 2552 | 57.3 | 58.9 |
| 45 | 2466 | 47.6 | 49.3 | 2492 | 56.0 | 57.5 |
| 60 | 2031 | 39.2 | 40.6 | 1958 | 44.0 | 45.2 |
| 90 | 1741 | 33.6 | 34.8 | 1246 | 28.0 | 28.8 |
| 150 | 1668 | 32.2 | 33.3 | 831 | 18.7 | 19.2 |
| 240 | 798 | 15.4 | 15.9 | 890 | 20.0 | 20.5 |

¹ after the 20 min residence time required for the disintegration;
² amount of pancrelipase microtablets added to 5 mL sodium bicarbonate: 72.54 mg × found Batch Lipase assay (71.5 Lipase USP units/mg) = 5187 USP units;
³ amount of pancrelipase microtablets added to 5 mL sodium bicarbonate: 59.34 mg × found Batch Lipase assay (75 Lipase USP units/mg) = 4451 USP units

TABLE 10b
Lipase activity of pancrelipase microtabs 5,000 USP
U in 8.4% sodium bicarbonate solution stored at 4° C.

| | RUN 1 | | | RUN 2 | | |
|---|---|---|---|---|---|---|
| | Total Lipase | % Lipase activity | % Lipase | Total lipase | % Lipase activity | % Lipase |
| Storage time | activity | of theor. total | activity | activity | of theor. total | activity |
| (min) | (USP U) | lipase ² | on t0 | (USP U) | lipase ³ | on t0 |
| Initial (t0) ¹ | 4265 | 92.3 | 100.0 | 4653 | 96.0 | 100.0 |
| 15 | 4006 | 86.7 | 93.9 | 4395 | 90.7 | 94.4 |
| 30 | 3748 | 81.1 | 87.9 | 4201 | 86.7 | 90.3 |
| 45 | 3554 | 76.9 | 83.3 | 3942 | 81.3 | 84.7 |
| 60 | 3360 | 72.7 | 78.8 | 3878 | 80.0 | 83.3 |
| 90 | 3296 | 71.3 | 77.3 | 3425 | 70.7 | 73.6 |
| 150 | 2973 | 64.3 | 69.7 | 3167 | 65.3 | 68.1 |
| 240 | 2714 | 58.7 | 63.6 | 2908 | 60.0 | 62.5 |

¹ after the 20 min residence time required for the disintegration;
² amount of pancrelipase microtablets added to 5 mL sodium bicarbonate: 64.62 mg × found batch lipase assay (71.5 lipase USP units/mg) = 4620 USP units;
³ amount of pancrelipase microtablets added to 5 mL sodium bicarbonate: 64.63 mg × found batch lipase assay (75 lipase USP units/mg) = 4847 USP units

TABLE 10c
Lipase activity of pancrelipase microtabs 5,000 lipase USP
units in 8.4% sodium bicarbonate solution stored at r.t./4° C.
(Mean values from Run 1 and 2)

| | Storage at r.t. | | Storage at 4° C. | |
|---|---|---|---|---|
| Storage | % Lipase activity | % Lipase | % Lipase activity | % Lipase |
| time | of theor. | activity | of theor. Total | activity |
| (min) | Total lipase | on t0 | lipase | on t0 |
| Initial | 96.9 | 100.0 | 94.2 | 100.0 |
| (t0)¹ | | | | |
| 15 | 73.8 | 76.1 | 88.7 | 94.2 |
| 30 | 57.3 | 59.2 | 83.9 | 89.1 |
| 45 | 51.8 | 53.4 | 79.1 | 84.0 |
| 60 | 41.6 | 42.9 | 76.4 | 81.1 |
| 90 | 30.8 | 31.8 | 71.0 | 75.4 |
| 150 | 25.4 | 26.3 | 64.8 | 68.9 |
| 240 | 17.7 | 18.2 | 59.4 | 63.1 |

¹After the 20 min residence time required for the disintegration.

The mixtures stored at 4° C. shows higher lipase stability than the ones stored at room temperature (˜60% vs ˜20% of residual lipase activity after 4 hours); in particular, for the sample stored at r.t. a nearly vertical drop in lipase activity (with less than 50% of the initial lipase activity left) is observed in the first hour.

Experiment 5.2. Lipase Activity of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*Disintegration Stage=20 Min at R.T.]

The contents of eight capsules are added to 5 mL of 8.4% sodium bicarbonate in a 15 mL beaker, without stirring. The sample is stored at r.t., bench top conditions, without stirring. Two independent samples are prepared (2A and 2B). After 20 minutes the pancrelipase/bicarbonate mixtures are stirred and a 150 μl aliquot is diluted to 100 mL water and the lipase activity is determined on 1 mL of this solution, following the lipase assay of the pancrelipase USP monograph (time 0). Immediately after the t0 sampling sample 2A is stored at room temperature, while sample 2B is kept at 4° C. Further 150 μl aliquots are withdrawn from both samples 2A and 2B after 15, 30, 45, 60, 120, and 240 min from t0, and immediately assayed for the lipase activity. The activity of lipase in the pancrelipase/sodium bicarbonate mixture is expressed as % of the total lipase activity calculated from the batch lipase assay found in the same run. The results are summarized in Table 11.

TABLE 11
Lipase activity of pancrelipase microtabs 40,000 Lipase
USP units in 8.4% sodium bicarbonate solution stored at
room temperature/4° C.

| | Storage at room | | | |
|---|---|---|---|---|
| | temperature | | Storage at 4° C. | |
| | Total | % Lipase | | % Lipase |
| | Lipase | activity | Total lipase | activity |
| Storage time | activity | of theor. total | activity | of theor. total |
| (min) | (USP U) | lipase² | (USP U) | lipase³ |
| Initial (t0)¹ | 23422 | 57.3 | 23366 | 58.7 |
| 15 | 35405 | 86.7 | 36642 | 92.0 |
| 30 | 33226 | 81.3 | 36642 | 92.0 |
| 45 | 27779 | 68.0 | 37174 | 93.3 |
| 60 | 23966 | 58.7 | 36642 | 92.0 |
| 120 | 16885 | 41.3 | 33987 | 85.3 |
| 240 | 9804 | 24.0 | 29739 | 74.7 |

¹After the 20 min residence time required for the disintegration;
²amount of pancrelipase microtablets added to 5 mL sodium bicarbonate: 544.69 mg × found batch lipase assay (75 Lipase USP units/mg) = 40852 USP units;
³amount of pancrelipase microtablets added to 5 mL sodium bicarbonate: 531.05 mg × found batch lipase assay (75 lipase USP units/mg) = 39829 USP units

The initial low value observed at t0 can be explained by an incomplete dissolution of the whole amount of microtablets in the 20 min disintegration stage. In both tested storage conditions the residual lipase activity measured is higher compared with the previous experiment performed on a individual dose unit.

Experiment 5.3. Lipase Activity of a Single Dose Unit of Pancrelipase Enzymes Microtablets with 5,000 Lipase USP U/Capsule (Zenpep® Microtabs 5,000 Lipase USP U/cps) Dissolved* in 5 mL 8.4% Sodium Bicarbonate Solution, R.T. vs 4° C. [*after 40 Min Disintegration Stage at R.T.]

The time of disintegration stage in this experiment is doubled compared to Experiment 2.1. The results are summarized in Table 12.

TABLE 12
Lipase stability of pancrelipase microtabs 5,000 lipase USP units
in 5 mL 8.4% sodium bicarbonate solution stored at room
temperature/4° C. (after 40 min disintegration stage at r.t.)

| | Storage at room | | | |
|---|---|---|---|---|
| | temperature | | Storage at 4° C. | |
| | Total | % lipase | | % lipase |
| | lipase | activity | Total lipase | activity |
| Storage time | activity | of theor. total | activity | of theor. total |
| (min) | (USP U) | lipase² | (USP U) | lipase³ |
| Initial (t0)¹ | 3962 | 87.6 | 4156 | 81.0 |
| 15 min | 3016 | 66.7 | 4156 | 81.0 |
| 30 min | 3844 | 85.0 | 3687 | 71.9 |
| 45 min | 1952 | 43.1 | 3553 | 69.3 |
| 60 min | 1597 | 35.3 | 3285 | 64.1 |
| 120 min | 1065 | 23.5 | 2950 | 57.5 |
| 240 min | 651 | 14.4 | 2615 | 51.0 |

¹After the 40 min residence time required for the disintegration;
²amount of pancrelipase microtablets (59.14 mg) × found batch lipase assay (76.5 Lipase USP units/mg) = 4524 USP units;
³amount of pancrelipase microtablets (67.04 mg) × found batch lipase assay (76.5 Lipase USP units/mg) = 5129 USP units

Due to the additional residence time in the bicarbonate medium the lipase stability is slightly worse when the disintegration time is prolonged.

Experiment 5.4. Lipase Activity of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*after 40 Min Disintegration Stage at R.T.]

This experiment follows the same procedure of Experiment 2.2, with the exception of the time of disintegration stage, which is doubled to ensure a complete disintegration of the higher amount of microtablets. The results are summarized in Table 13.

TABLE 13
Lipase activity of pancrelipase microtabs 40,000 lipase USP units
in 5 mL 8.4% sodium bicarbonate solution stored at room
temperature/4° C. (after 40 min disintegration stage).

| | Storage at room | | | |
|---|---|---|---|---|
| | temperature | | Storage at 4° C. | |
| | Total | % Lipase | | % lipase |
| | lipase | activity | Total lipase | activity |
| Storage time | activity | of theor. total | activity | of theor. total |
| (min) | (USP U) | lipase² | (USP U) | lipase³ |
| Initial (t0)¹ | 31276 | 82.9 | 29855 | 78.9 |
| 15 min | 32269 | 85.5 | 32343 | 85.5 |
| 30 min | 28297 | 75.0 | 33339 | 88.2 |
| 45 min | 22836 | 60.5 | 32841 | 86.8 |
| 60 min | 19361 | 51.3 | 31348 | 82.9 |
| 120 min | 15390 | 40.8 | 30851 | 81.6 |
| 240 min | 8936 | 23.7 | 27865 | 73.7 |

¹After the 40 min residence time required for the disintegration;
²amount of pancrelipase microtablets (496.44 mg) × found batch lipase assay (76 lipase USP units/mg) = 37729 USP units;
³amount of pancrelipase microtablets (497.59 mg) × found batch lipase assay (76 lipase USP units/mg) = 37817 USP units

As already observed in previous tests, satisfactory lipase stability is observed in sample suspensions stored at 4° C. (about 74% of residual activity after four-hour storage). No significant differences in Lipase stability are observed by increasing the disintegration time from 20 to 40 min for the 40,000 lipase USP units pooled samples, as in the 20 min stage part of the microtablets are not completely disintegrated.

Experiment 5.5. Protease Activity of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*after 40 Min Disintegration Stage at R.T.]

The contents of eight capsules, corresponding to approx. 40,000 Lipase USP units, are added to 5 mL of 8.4% sodium bicarbonate in a 15 mL beaker, without stirring. The sample is stored at r.t., bench top conditions, without stirring. Two independent samples are prepared (2A and 2B). After 40 min the pancrelipase/bicarbonate mixtures are stirred and a 140 μl aliquot is diluted to 100 mL cold pH 7.5 buffer. 1 mL of this solution is further diluted to 20 mL with cold pH 7.5 buffer. The protease activity is determined according to the compendia procedure described in the pancrelipase USP monograph (time 0). Immediately after taking the t0 aliquot sample 2A is stored at room temperature, while sample 2B is kept at 4° C. Further 140 μl aliquots are withdrawn from both samples 2A and 2B after 30, 60, 120, 240 min from t0, and immediately assayed for the protease activity. The activity of protease in the pancrelipase/sodium bicarbonate mixture is expressed as % of the total protease activity calculated from the batch protease assay (177 USP U/mg). The results are summarized in Table 14.

TABLE 14
Protease activity of pancrelipase microtabs 40,000 lipase USP units
in 5 mL 8.4% sodium bicarbonate solution stored at room
temperature/4° C. (after 40 min disintegration stage)

| | Storage at room | | | |
|---|---|---|---|---|
| | temperature | | Storage at 4° C. | |
| | Total | % Protease | Total | % Protease |
| | protease | activity | protease | activity |
| Storage time | activity | of theor. total | activity | of theor. total |
| (min) | (USP U) | protease² | (USP U) | protease³ |
| Initial (t0)¹ | 72115 | 81.4 | 74141 | 81.9 |
| 30 min | 67107 | 75.7 | 77209 | 85.3 |
| 60 min | 78626 | 88.7 | 78232 | 86.4 |
| 120 min | 71114 | 80.2 | 91526 | 101.1 |
| 240 min | 77624 | 87.6 | 98685 | 109.0 |

¹After the 40 min residence time required for the disintegration;
²amount of pancrelipase microtablets (500.8 mg) × batch protease assay (177 protease USP units/mg) = 88642 USP units;
³amount of pancrelipase microtablets (511.32 mg) × batch protease assay (177 Protease USP units/mg) = 90504 USP units

In both storage conditions protease displays a satisfactory stability over four hours, with residual activity between 75.7 and 88.7% for sample solution stored at r.t. and between 81.9 and 109% for sample solutions stored at 4° C.

Experiment 5.6. Amylase Activity of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*after 40 Min Disintegration Stage at R.T.].

The contents of eight capsules corresponding to approx. 40,000 lipase USP units, are added to 5 mL of 8.4% sodium bicarbonate in a 15 mL beaker, without stirring. The sample is stored at r.t., bench top conditions, without stirring. Two independent samples are prepared (2A and 2B). After 40 minutes the pancrelipase/bicarbonate mixtures are stirred and a 270 μl aliquot is diluted to 5 mL cold pH 6.8 amylase buffer. 1 mL of this solution is further diluted to 20 mL with cold pH 6.8 amylase buffer. The amylase activity is determined according to the compendia procedure described in the pancrelipase USP monograph (time 0). Immediately after taking the t0 aliquot sample 2A is stored at room temperature, while sample 2B is kept at 4° C. Further 270 μl aliquots are withdrawn from both samples 2A and 2B after 60 and 120 min from t0, and immediately assayed for the amylase activity. The stability of amylase in the pancrelipase/sodium bicarbonate mixture is expressed as % of the total amylase activity calculated from the batch amylase assay (238 USP U/mg). Results are summarized in Table 15.

TABLE 15
Amylase stability of pancrelipase microtabs 40,000 lipase USP units
in 5 mL 8.4% sodium bicarbonate solution stored at room
temperature/4° C. (after 40 min disintegration stage).

| | Storage at room | | | |
|---|---|---|---|---|
| | temperature | | Storage at 4° C. | |
| | Total | % Amylase | Total | % Amylase |
| | amylase | activity | amylase | activity |
| Storage time | activity | of theor. total | activity | of theor. total |
| (min) | (USP U) | amylase² | (USP U) | amylase³ |
| Initial (t0)¹ | 111071 | 89.1 | 99843 | 79.8 |
| 60 min | 80684 | 64.7 | 94063 | 75.2 |
| 120 min | 58679 | 47.1 | 99318 | 79.4 |

¹After the 40 min residence time required for the disintegration;
²amount of pancrelipase microtablets (523.92 mg) × batch amylase assay (238 Amylase USP units/mg) = 124693 USP units;
³amount of pancrelipase microtablets (525.49 mg) × batch amylase assay (238 amylase USP units/mg) = 125067 USP units

The amylase activity is improved in sample solution stored at 4° C. (between 75 and 80% of the theoretical enzyme activity over the tested time period) in comparison with the sample solution stored at r.t.

Experiment 5.7. Lipase Activity of a Single Dose Unit of Pancrelipase Enzymes Microtablets with 5,000 Lipase USP U/Capsule (Zenpep® Microtabs 5,000 Lipase USP U/cps) Dissolved* in 5 mL 13% Sodium Bicarbonate Saturated Solution, R.T. vs 4° C. [*after 20 Min Disintegration Stage at R.T.]

This experiment follows the same procedure described in the Experiment 2.1, the only difference being the concentration of sodium bicarbonate solution utilized (13%). The stability of lipase in the pancrelipase/sodium bicarbonate mixture is expressed as % of the total lipase activity calculated from the batch lipase assay found in the same run. The results are summarized in Table 16.

TABLE 16
Lipase stability of pancrelipase microtabs, approx 5,000 USP units
in 5 mL 13% sodium bicarbonate saturated solution stored at room
temperature/4° C. (after 20 min disintegration stage)

| | Storage at room | | | |
|---|---|---|---|---|
| | temperature | | Storage at 4° C. | |
| | Total | % Lipase | | % Lipase |
| | lipase | activity | Total lipase | activity |
| Storage time | activity | of theor. total | activity | of theor. total |
| (min) | (USP U) | lipase² | (USP U) | lipase³ |
| Initial (t0)¹ | 4343 | 101.3 | 4629 | 98.7 |
| 15 min | 3429 | 80.0 | 4566 | 97.3 |
| 30 min | 2743 | 64.0 | 4379 | 93.3 |
| 45 min | 2229 | 52.0 | 4003 | 85.3 |
| 60 min | 1829 | 42.7 | 3753 | 80.0 |
| 120 min | 1200 | 28.0 | 3190 | 68.0 |
| 240 min | 800 | 18.7 | 2690 | 57.3 |

¹After the 20 min residence time required for the disintegration;
²amount of pancrelipase microtablets (57.15 mg) × found batch lipase assay (75 lipase USP units/mg) = 4286 USP units;
³amount of pancrelipase microtablets (62.55 mg) × found batch lipase assay (75 lipase USP units/mg) = 4691 USP units

The lipase stability profiles observed in the two storage conditions are almost completely superimposable with those obtained with the 8.4% sodium bicarbonate solution meaning that the increased concentration of the alkaline substance does not affect the stability of this enzyme.

Experiment 5.8. Lipase Activity of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*after 45 Min Disintegration Stage at 4° C.]

This experiment follows the same procedure described in experiment 2.2, the only difference being the disintegration stage, performed at 4° C. for 45 min. The stability of lipase in the pancrelipase/sodium bicarbonate mixture is expressed as % of the total lipase activity calculated from the batch Lipase Assay found in the same run. The results are summarized in Table 17.

TABLE 17
lipase activity of pancrelipase microtabs 40,000U in 5 mL
8.4% sodium bicarbonate solution stored at 4° C.
(after 45 min disintegration stage at 4° C.)

Storage at 4° C.

| | | % Lipase activity |
|---|---|---|
| | Total lipase activity | of theor. total |
| Storage time (min) | (USP U) | lipase² |
| Initial (t0)¹ | 33092 | 84.2 |
| 15 min | 37229 | 94.7 |
| 30 min | 33610 | 85.5 |
| 45 min | 36195 | 92.1 |
| 60 min | 34127 | 86.8 |
| 120 min | 32575 | 82.9 |
| 240 min | 28439 | 72.4 |

¹After the 20 min residence time required for the disintegration;
²amount of pancrelipase microtablets (517.07 mg) × found batch lipase assay (76 lipase USP units/mg) = 39297 USP units.

The lipase stability profile observed is equivalent with the one obtained with a 40 min disintegration stage at r.t.: the 40 min disintegration stage at r.t., for 40,000 lipase USP units pooled samples, does not affect the stability of this enzyme.

Experiment 5.9. Lipase Activity of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 25 mL 8.4% Sodium Bicarbonate Solution [*after 20 Min Disintegration Stage at R.T.]

The contents of eight capsules corresponding to approx. 40,000 Lipase USP units, are added to 25 mL of 8.4% sodium bicarbonate in a 50 mL beaker, without stirring. The sample is stored at r.t., bench top conditions. Two independent samples are prepared (2A and 2B). After 20 min the pancrelipase/bicarbonate mixtures are stirred and a 150 μl aliquot is diluted to 20 mL with water and the mixtures are stirred and a 150-μl aliquot is diluted to 20 mL water and the lipase activity is determined on 1 mL of this solution according to the compendia procedure in the Pancrelipase USP monograph (time 0). Immediately after taking the t0 aliquot, sample 2A is stored at room temperature, while sample 2B is kept at 4° C. Further 150 μl aliquots are withdrawn from both solutions 2A and 2B after 15, 30, 45, 60, 120, and 240 minutes from t0, and immediately assayed for the lipase activity. The stability of lipase in the pancrelipase/sodium bicarbonate mixture is expressed as % of the total lipase activity calculated from the batch lipase assay found in the same run. The results are summarized in Table 18.

TABLE 18
Lipase activity of pancrelipase microtabs, approx 40,000 lipase
USP units in 25 mL 8.4% sodium bicarbonate solution stored
at room temperature/4° C. (after 20 min disintegration stage)

| | Storage at room temperature | | Storage at 4° C. | |
|---|---|---|---|---|
| | | % Lipase | | % Lipase |
| | Total lipase | activity | Total Lipase | activity of |
| Storage time | activity | of theor. total | activity | theor. total |
| (min) | (USP U) | lipase² | (USP U) | lipase³ |
| Initial (t0)¹ | 36335 | 99.3 | 35688 | 90.1 |
| 15 min | 35850 | 98.0 | 29390 | 74.2¹¹ |
| 30 min | 28099 | 76.8 | 37262 | 94.0 |
| 45 min | 25192 | 68.9 | 35163 | 88.7 |
| 60 min | 22285 | 60.9 | 34638 | 87.4 |
| 120 min | 17925 | 49.0 | 33064 | 83.4 |
| 240 min | 15018 | 41.1 | 30964 | 78.1 |

¹after the 20 min residence time required for the disintegration;
²amount of pancrelipase microtablets (484.46 mg) × found batch lipase assay (75.5 lipase USP units/mg) = 36577 USP units;
³amount of pancrelipase microtablets (524.82 mg) × found batch lipase assay (75.5 lipase USP units/mg) = 39624 USP units.

The lipase stability of the 25 mL sample solution stored at 4° C. displays the same profile observed in the same storage condition for the same amount of microtablets disintegrated in 5 mL of bicarbonate medium, while the 25 mL sample solution stored at r.t. shows an improved stability compared with the corresponding sample at lower volume (˜40% of residual enzyme activity vs ˜23%, after four hours).

### Experiment 5.10. Lipase Stability of Pancrelipase Powder (40,000 Lipase USP Units) Dissolved in 25 mL 8.4% Sodium Bicarbonate Solution

An amount of pancrelipase powder, corresponding to approx. 40,000 Lipase USP units is added to 25 mL of 8.4% sodium bicarbonate in a 50 mL beaker and briefly stirred (2 min) to get a homogeneous mixture. Two independent samples are prepared (2A and 2B). A 150 μl aliquot is diluted to 20 mL water and the lipase activity is determined on 1 mL of this solution, with the compendia procedure of the Pancrelipase USP monograph (time 0). Immediately after taking the t0 aliquot sample 2A is stored at room temperature, while sample 2B is kept at 4° C. Further 150 μl aliquots are withdrawn from both samples 2A and 2B after 15, 45, 60, 120, and 240 min from t0, and immediately assayed for the lipase activity. The stability of lipase in the pancreatin powder/sodium bicarbonate mixture is expressed as % of the total lipase activity calculated from the batch lipase assay found in the same run (119 USP U/mg). The results are summarized in Table 19.

TABLE 19
Lipase activity of pancrelipase powder (approx. 40,000 lipase
USP units) in 25 mL 8.4% sodium bicarbonate solution stored at
room temperature/4° C.

| | Storage at room temperature | | Storage at 4° C. | |
|---|---|---|---|---|
| | | % Lipase | | % Lipase |
| Storage | Total lipase | activity | Total lipase | activity |
| time | activity | of theor. total | activity | of theor. total |
| (min) | (USP U) | lipase¹ | (USP U) | lipase² |
| Initial (t0) | 39747 | 97.5 | 38978 | 95.0 |
| 15 min | 29811 | 73.1 | 35874 | 87.4 |
| 30 min | 26041 | 63.9 | 33459 | 81.5 |
| 45 min | 22958 | 56.3 | 31390 | 76.5 |
| 60 min | 19531 | 47.9 | 31390 | 76.5 |
| 120 min | 15419 | 37.8 | 30700 | 74.8 |
| 240 min | 10622 | 26.1 | 25526 | 62.2 |

¹amount of pancrelipase powder (342.65 mg) × found batch lipase assay (119 lipase USP units/mg) = 40775 USP units;
²amount of pancrelipase powder (344.94 mg) × found batch lipase assay (119 lipase USP units/mg) = 41048 USP units

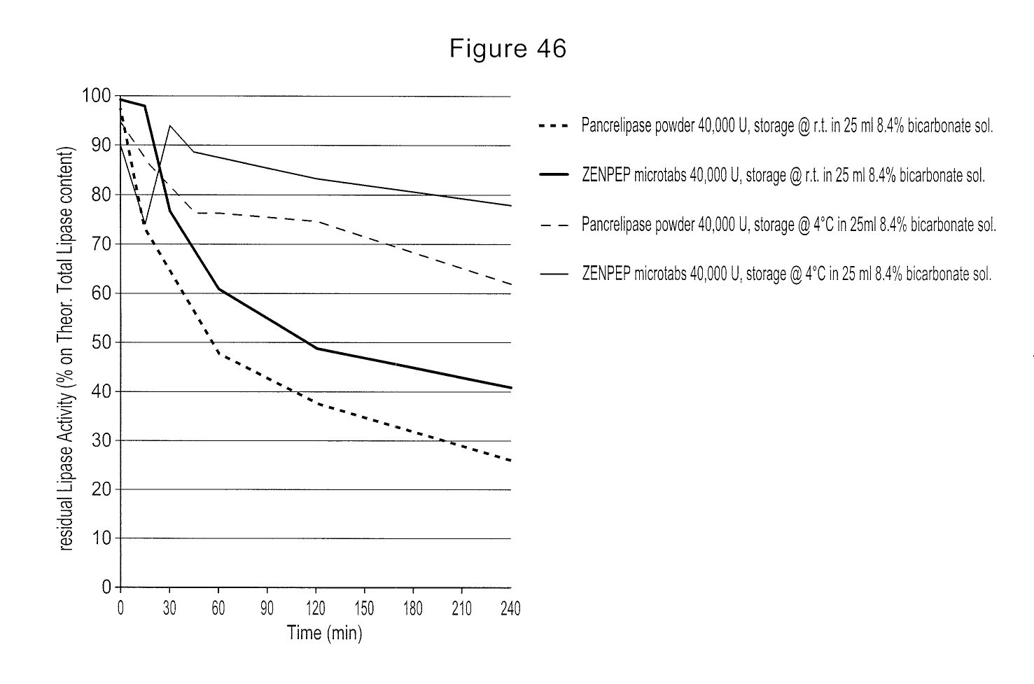

The lipase stability profile of pancrelipase powder, same lot contained in the pancrelipase microtabs used in the other experiments, is remarkably lower (14-16% less residual lipase activity at the 240 min endpoint in both storage conditions) than the one observed for the enteric coated pancrelipase microtabs. The comparison of Lipase stability profiles of disintegrated microtablets vs. pancrelipase powder, in 25 mL 8.4% sodium bicarbonate solutions is shown in FIG. 46.

Experiment 6. Determination of Lipase Activity in Liquid Nutritional Composition Added with the Pancrelipase Enzymes Beads/Bicarbonate Solution (Beads Disintegrated in Weakly Basic Solution).
Experiment 6.1. Lipase Activity in Liquid Nutritional Composition of Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*Disintegration Stage=20 Min at R.T.]

The contents of eight capsules, corresponding to 40,000 lipase USP units, are added to 5 mL of 8.4% sodium bicarbonate in a 30 mL beaker, without stirring. After 20 minutes the pancrelipase/bicarbonate mixture is stirred and poured into the bottle containing 2000 mL of liquid nutritional composition (see the composition above); (pH of the liquid nutritional composition “as is”=6.423; pH of the liquid nutritional composition+5 mL 8.4% sodium bicarbonate solution containing dissolved microtabs=6.724); the container is closed and briefly shaken. A 3 mL aliquot of the resulting mixture is poured into a 50 mL volumetric flask and diluted to volume with cold water; the solution is briefly shaken (theoretical lipase concentration=12 USP units/ml). This is the T0 sample. 1 mL of the T0 sample is immediately assayed, following the lipase assay of the Pancrelipase USP monograph to determine the lipase activity. The container is closed and stored at r.t. without stirring; before taking each aliquot the mixture is briefly shaken. The procedure is repeated after 15, 30, 60, 120, 240, and 360 min, by sampling at any time point 3 mL aliquots of the mixture stored at r.t. and following the dilution and analysis described for the T0 sample. The stability of lipase in the liquid nutritional composition (200 ml) added with pancrelipase/sodium bicarbonate mixture is expressed as % of the total lipase activity calculated from the batch lipase assay mean value obtained in the experiments 2.1-2.4, 2.7-2.9 (75.1 lipase USP U/mg). The results are summarized in Table 20.

TABLE 20
Lipase activity in 200 mL of liquid nutritional composition added
with 5 mL 8.4% bicarbonate solution containing the dissolved
pancrelipase enzymes, at r.t.

Storage at room temperature

| | Total lipase activity | % Lipase activity |
|---|---|---|
| Storage time (min) | (USP U)² | of theor. total lipase |
| Initial (t0)¹ | 35888 | 91.9 |
| 15 | 34327 | 87.9 |
| 30 | 35367 | 90.5 |
| 60 | 36928 | 94.5 |
| 120 | 37968 | 97.2 |
| 240 | 38488 | 98.5 |
| 360 | 38488 | 98.5 |

¹After the 20 min residence time required for the disintegration;
²amount of pancrelipase microtablets added to 5 mL sodium bicarbonate: 520.11 mg × batch lipase assay (75.1 lipase USP units/mg) = 39060 USP units.

The lipase activity remained stable in the time range considered; the slight decrease observed in the first time points (t0-30 min) can be explained with incomplete dissolution of the whole amount of microtablets in the 20 min disintegration stage; however, all the remaining residues are likely completely dissolved after the first-hour in the liquid meal. No lipase degradation is observed once the pancrelipase enzymes/bicarbonate mixture is poured into the tested liquid nutritional composition: lipase remains stable for at least 6 hours after preparation.

Experiment 6.2. Lipase Stability in Liquid Nutritional Composition of Pancrelipase Microtabs (Approx 40,000 lipase USP Units) Dissolved* in 5 mL 8.4% Sodium Bicarbonate Solution [*after 45 Min Disintegration Stage at 4° C.]

The contents of eight capsules, corresponding to approx. 40,000 lipase USP units, are added to 5 mL of 8.4% sodium bicarbonate in a 30 mL beaker, without stirring, and stored at 4° C. After 45 min the pancrelipase/bicarbonate mixture is stirred and poured into the bottle of liquid nutritional composition; the container is closed and briefly shaken. A 3 mL aliquot of the resulting mixture is poured into a 50 mL volumetric flask and diluted to volume with cold water; the solution is briefly shaken. This is the T0 sample. 1 mL of the T0 sample is immediately assayed to determine the lipase activity with the compendia procedure in the pancrelipase USP monograph. The container is closed and stored at r.t. without stirring; before taking each aliquot the mixture is briefly shaken. The same procedure is repeated after 15, 30, 60, 120, and 240 min, by sampling at any time point 3 mL aliquots of the mixture stored at r.t. and following the dilution and analysis described for the T0 sample. The stability of lipase in the test liquid nutritional composition added with pancrelipase/sodium bicarbonate solution is expressed as % of the total lipase activity calculated from the mean of batch lipase assay values obtained in the previous experiments 2.1-2.4, 2.7-2.9 (75.1 lipase USP U/mg). The results are summarized in Table 21.

TABLE 21
Lipase activity in liquid nutritional composition at r.t., 200 mL added
with 5 mL 8.4% bicarbonate solution containing the dissolved
40,000 lipase USP units pancrelipase microtabs (45 min
disintegration stage at 4° C.)

Storage at room temperature

| | Total lipase activity | % Lipase activity |
|---|---|---|
| Storage time (min) | (USP U)² | of theor. total lipase |
| Initial (t0)¹ | 34042 | 93.2 |
| 15 min | 34529 | 94.5 |
| 30 min | 35988 | 98.5 |
| 60 min | 37447 | 102.5 |
| 120 min | 37933 | 103.9 |
| 240 min | 38906 | 106.5 |

¹After the 45 min residence time in sodium bicarbonate solution required for the disintegration of microtabs;
²amount of pancrelipase microtablets (486.32 mg) × batch lipase assay (75.1 lipase USP units/mg) = 36,523 USP units

The lipase activity remained stable in the tested liquid nutritional composition over four hours. During the experiment a gradual increase of enzyme activity is observed; it may be due to an enzyme conformational change (associated with increased activity) induced by the components of the liquid meal. Except for the higher lipase activity measured, the stability profile of this experiment is similar to the previous one (liquid nutritional composition added with the same amount of microtablets dissolved after a shorter disintegration time in 5 mL 8.4% bicarbonate solution).

Experiment 6.3. Protease Activity in Liquid Nutritional Composition of a Pooled Sample of Eight Units of Pancrelipase Enzymes Microtablets with 5,000 Lipase USP U/Capsule (Zenpep® Microtabs 5,000 Lipase USP U/Cps) (5,000 Lipase USP U/Capsule=40,000 Lipase USP U) Dissolved* in 5 mL 8.4% Sodium Bicarbonate Solution [*Disintegration Stage=45 Min at 4° C.].

The contents of eight capsules, corresponding to approx. 40,000 lipase USP units, are added to 5 mL of 8.4% sodium bicarbonate in a 30 mL beaker, without stirring, and stored at 4° C. After 45 min the pancrelipase/bicarbonate mixture is stirred and poured into the bottle of the liquid nutritional composition; the container is closed and briefly shaken. A 2.8 mL aliquot of the resulting mixture is poured into a 20 mL volumetric flask and diluted to volume with cold pH 7.5 buffer; 1 mL of this solution is further diluted to 50 mL with cold pH 7.5 buffer. The protease activity is determined with the compendia procedure of the pancrelipase USP monograph (time 0). The container is closed and stored at r.t. without stirring; before taking each aliquot the mixture is briefly shaken. The same procedure is repeated after 30, 60, 120, and 240 min, by sampling at any time point 2.8 mL aliquots of the mixture stored at r.t. and following the dilution and analysis described for the T0 sample. The stability of protease in the tested liquid meal added with pancrelipase/sodium bicarbonate mixture is expressed as % of the total protease activity calculated from the batch protease assay (177 USP U/mg). The results are summarized in Table 22.

TABLE 22
Protease activity in liquid meal at r.t., 200 mL added with 5 mL
8.4% bicarbonate solution containing the dissolved pancrelipase
microtabs, approx 40,000 lipase USP units (45 min disintegration
stage at 4° C.)

Storage at room temperature

| | Total protease | % Protease activity |
|---|---|---|
| Storage | activity | of theor. total |
| time (min) | (USP U)² | protease |
| Initial (t0)¹ | 69018 | 74.6 |
| 30 min | 89409 | 96.6 |
| 60 min | 89409 | 96.6 |
| 120 min | 94638 | 102.3 |
| 240 min | 96729 | 104.5 |

¹After the 45 min residence time in sodium bicarbonate solution required for the disintegration of microtabs;
²amount of pancrelipase microtablets (522.86 mg) × batch protease assay (177 protease USP units/mg) = 92,546 USP units.

For over four hours protease residual activity is very close (or slightly greater) to 100% of theoretical total enzyme content.

Experiment 6.4. Amylase Activity in Liquid Nutritional Meal of a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 5 mL 8.4% Sodium Bicarbonate Solution [*after 45 Min Disintegration Stage at 4° C.].

The contents of eight capsules, corresponding to 40,000 lipase USP units, are added to 5 mL of 8.4% sodium bicarbonate in a 30 mL beaker, without stirring, and stored at 4° C. After 45 min the pancrelipase/bicarbonate mixture is stirred and poured into the bottle of the liquid meal; the container is closed and briefly shaken. A 2.2 mL aliquot of the resulting mixture is poured into a 20 mL volumetric flask, diluted to volume with cold pH 6.8 amylase buffer and shaken. The amylase activity is determined with the compendia procedure of the pancrelipase USP monograph (time 0). The container is closed and stored at r.t. without stirring; before taking each aliquot the mixture is briefly shaken. The same procedure is repeated after 60 and 120 min, by sampling at any time point 2.2 mL aliquots of the mixture stored at r.t. and following the dilution and analysis described for the T0 sample. The stability of amylase in the tested meal added with pancrelipase/sodium bicarbonate mixture is expressed as % of the total amylase activity calculated from the batch amylase assay (238 USP U/mg). The results are summarized in Table 23.

TABLE 23
Amylase stability in liquid nutritional meal at r.t., 200 mL added
with 5 mL 8.4% bicarbonate solution containing the dissolved
pancrelipase microtabs, approx 40,000 lipase USP units (45 min
disintegration stage at 4° C.).

Storage at room temperature

| | Total amylase | % amylase activity |
|---|---|---|
| Storage | activity | of theor. total |
| time (min) | (USP U)² | amylase |
| Initial (t0)¹ | 109006 | 87.8 |
| 60 min | 100661 | 81.1 |
| 120 min | 93359 | 75.2 |

¹After the 45 min residence time in sodium bicarbonate solution required for the disintegration of microtabs;
²amount of pancrelipase microtablets (521.56 mg) × batch amylase assay (238 amylase USP units/mg) = 124,131 USP units

The amylase activity displays a gradual reduction over two hours in tested liquid meal (from 88 to 75% of theoretical total enzyme content), thus the decrease is less pronounced than in sodium bicarbonate solution.

Experiment 6.5. Lipase Stability in Liquid Nutritional Meal Plus a Pooled Sample of Eight Units of Pancrelipase Enzymes Microtablets with 5,000 Lipase USP U/Capsule (Zenpep® Microtabs 5,000 Lipase USP U/cps) (5,000 Lipase USP U/Capsule=40,000 Lipase USP U) Dissolved* in 25 mL 8.4% Sodium Bicarbonate Solution [*after 20 Min Disintegration Stage at R.T.]

The contents of eight capsules, corresponding to 40,000 lipase USP units, are added to 25 mL of 8.4% sodium bicarbonate in a suitable beaker, without stirring. After 20 min the pancrelipase/bicarbonate mixture is stirred and poured into the bottle of the liquid meal; the container is closed and briefly shaken. A 3.5 mL aliquot of the resulting mixture is poured into a 50 mL volumetric flask and diluted to volume with cold water; the solution is briefly shaken. This is the T0 sample. 1 mL of the T0 sample is immediately assayed to determine the lipase activity, with the compendia procedure of pancrelipase USP monograph. The container is closed and stored at r.t. without stirring; before taking each aliquot the mixture is briefly shaken. The same procedure is repeated after 15, 30, 60, 120, 240, and 360 min, by sampling at any time point 3.5 mL aliquots of the mixture stored at r.t. and following the dilution and analysis described for the T0 sample. The stability of lipase in the tested liquid nutritional composition added with pancrelipase/sodium bicarbonate mixture is expressed as % of the total lipase activity calculated from the mean of batch lipase assay values obtained in the experiments 2.1-2.4, 2.7-2.9 (75.1 lipase USP U/mg). The results are summarized in Table 24.

TABLE 24
Lipase activity in liquid nutritional composition at r.t., 200 mL added
with 25 mL 8.4% bicarbonate solution containing the dissolved
pancrelipase microtabs, approx 40,000 lipase USP units (20 min
disintegration stage)

Storage at room temperature

| Storage | Total lipase activity | % Lipase activity |
|---|---|---|
| time (min) | (USP U)² | of theor. total lipase |
| Initial (t0)¹ | 32732 | 85.2 |
| 15 min | 33244 | 86.6 |
| 30 min | 34266 | 89.2 |
| 60 min | 34778 | 90.5 |
| 120 min | 36824 | 95.9 |
| 240 min | 37335 | 97.2 |
| 360 min | 37847 | 98.5 |

¹After the 20 min residence time in sodium bicarbonate solution required for the disintegration of microtabs;
²amount of pancrelipase microtablets (511.44 mg) × batch lipase assay (75.1 lipase USP units/mg) = 38,409 USP units

With the exception of t0, the lipase stability profile is almost completely superimposable with the one obtained in the same liquid meal added with the product dissolved in a smaller volume (5 mL) of 8.4% sodium bicarbonate solution, meaning that the increased volume of bicarbonate medium does not affect the stability of this enzyme.

Although illustrated and described above with reference to certain specific embodiments and examples, the present invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges.

1. A process for the preparation of a predigested nutritional formula comprising mixing digestive enzymes or enzyme solution thereof and a liquid nutritional composition comprising a mixture of carbohydrates, lipids, proteins and water to form the predigested nutritional formula.

2.-4. (canceled)

5. The process of claim 1 wherein the digestive enzymes are in the form of pancrelipase beads.

6. The process of claim 1 wherein the digestive enzymes are in the form of enterically-coated pancrelipase beads.

7. (canceled)

8. The process of claim 1 wherein the mixing is conducted by mechanical blending of the pancrelipase beads and liquid nutritional composition until the mixture is homogenized.

9. The process of claim 1 wherein the enzyme solution thereof is prepared by suspending enterically coated pancrelipase beads in a pharmaceutically acceptable weakly basic solution.

10. The process of claim 1, wherein mixing is carried out continuously at mixing speed of between about 12,500 and about 18,000 rpm for about 1 to about 2 minutes at about room temperature.

11. The process of claim 1 wherein the mixture resulting from the mixing is then combined with a further portion of the liquid nutritional composition.

12. (canceled)

13. (canceled)

14. The process of claim 9, wherein the mixture of pancrelipase beads in weakly basic solution is held for about 20 minutes to about 120 minutes before it is added to the liquid nutritional composition.

15. The process of claim 9, wherein the pharmaceutically acceptable weakly basic solution comprises at least one alkaline substance, one amino acid, or a mixture thereof.

16. The process of claim 15, wherein the alkaline substance is selected from the group consisting of alkali and alkaline earth metal hydroxides, carbonates, bicarbonates, sulphates, phosphates, oxides and tris-(hydroxymethyl)-aminomethane (THAM) and mixture thereof.

17. (canceled)

18. The process of claim 16, wherein the alkaline substance is sodium bicarbonate and the sodium bicarbonate concentration ranges from about 0.65 to about 13% weight/volume.

19. The process of claim 9 or wherein the pH of the pharmaceutically acceptable weakly basic solution ranges from about 7.5 to about 8.5.

20. The process of claim 9, wherein the mixture of pancrelipase beads in weakly basic solution is held below room temperature before it is added to the liquid nutritional composition.

21. The process of claim 5 wherein the pancrelipase beads are in an amount equivalent to approximately between about 2,000 and about 4,000 Lipase USP units per g fat of the liquid nutritional composition.

22. The process of claim 1, wherein the caloric content of the liquid nutritional composition comprises about 28-90% carbohydrates, about 1-55% fat, and about 4-32% protein total.

23. The process of claim 1, wherein the enzymes used are in the form of granules, tablets, spheres, minitablets, microtablets, microparticles, microspheres, microcapsules or micropellets.

24. The process of claim 1 wherein the enzymes have a total of about 3,000, about 4,200, about 5,000, about 6,000, about 10,000, about 10,500, about 15,000, about 16,800, about 20,000, about 21,000, about 24,000, or 25,000 USP, lipase units or multiple thereof, or about 5,000 or about 30,000 PhEur lipase units or multiple thereof.

25. The process of claim 1, wherein a therapeutically effective amount of enzymes is added.

26. A predigested nutritional formula prepared by the process of claim 1, wherein the enzymes have a lipase activity as percentage of the units of lipase activity added to the liquid nutritional composition above about 85%.

27. The predigested nutritional formula of claim 26, wherein the enzymes have a lipase activity as percentage of the units of lipase activity added to the liquid nutritional composition above about 90%.

28. The predigested nutritional formula of claim 26, wherein the enzymes have a lipase activity as percentage of the units of lipase activity added to the liquid nutritional composition above about 95%.

29. The predigested nutritional formula of claim 26, wherein the enzymes have a mean lipase activity as percentage of the units of lipase activity added to the liquid nutritional composition greater than about 95% after about 360 minutes of storage time at about room temperature.

30. (canceled)

31. The predigested nutritional formula of claim 26, wherein the lipids comprise triglycerides, wherein at about 1 hour after preparation the amount of said triglycerides are about 45% of the initial value.

32. The predigested nutritional formula of claim 26, wherein the lipids comprise triglycerides, wherein at about 8 hours after preparation the amount of said triglycerides are about 30% of the initial value.

33. The pancrelipase predigested nutritional formula of claim 26, wherein the lipids comprise triglycerides, wherein at about 1 hour after preparation the percentage of acyl chains released from the triglycerides is about 16%.

34. The predigested nutritional formula of claim 31, wherein the lipids comprise triglycerides, wherein at about 1 hour after preparation the percentage of the triglyceride acyl chains converted into free fatty acid acyl chains and into monoglyceride acyl chains is about 28%.

35. The predigested nutritional formula of claim 26, wherein the lipids comprise triglycerides, wherein at about 8 hours after preparation the percentage of acyl chains released from the triglycerides is about 28%.

36. The predigested nutritional formula of claim 32, wherein the lipids comprise triglycerides, wherein at about 8 hours after preparation the percentage of the triglyceride acyl chains converted into free fatty acid acyl chains and into monoglyceride acyl chains is about 36%.

37. A method of administration to a patient in need thereof of a predigested nutritional formula comprising the steps of:
a) transferring the predigested nutritional formula of claim 26 to a dispensing bag;
b) dispensing the pancrelipase predigested nutritional formula from the bag to the patient through an enteral tube, gauge gastrostomy tube, nasogastric tube or jejunal tube.

38. (canceled)

39. A method of administration to pediatric or adult patients of an predigested nutritional formula comprising the step of:
a. pouring a portion of a liquid nutritional composition comprising a mixture of carbohydrates, lipids, proteins and water into a blender;
b. adding the total amount of the dose of pancrelipase beads into the blender and closing the blender;
c. mixing the resultant mixture continuously at mixing speed of between about 15,000 and about 16,500 rpm for about 1-2 minutes at about room temperature;
d. combining the blend obtained in step c) with a second portion of the liquid nutritional composition to achieve a final volume of the total predigested nutritional formula;
e. transferring the predigested nutritional formula of step d) into a dispensing bag;
f. shaking the bag gently before starting the feeding;
g. starting the feeding through an enteral tube.

40. (canceled)

41. A kit for the preparation of a predigested nutritional formula for enteral feeding comprising:
a. a liquid nutritional composition comprising an mixture of carbohydrates, lipids, proteins and water;
b. pancrelipase beads.

42. (canceled)

43. (canceled)
